(19) United States
(12) Patent Application Publication
(10) Pub. No.: US 2020/0224277 A1
(43) Pub. Date: Jul. 16, 2020

(54) CELL TYPE IDENTIFICATION METHOD AND SYSTEM THEREOF

(71) Applicant: MAO YING GENETECH INC., Taipei City (TW)

(72) Inventor: PEI-ING HWANG, Taipei City (TW)

(21) Appl. No.: 16/631,165

(22) PCT Filed: Jul. 16, 2018
(86) PCT No.: PCT/CN2018/095805

§ 371 Date: Jan. 15, 2020

Related U.S. Application Data
(60) Provisional application No. 62/533,145, filed on Jul. 17, 2017.

(51) Int. Cl. C12Q 1/6886; C12Q 1/6881; C12Q 1/6851; C12Q 1/686; G16B 20/00; G16B 25/10

(52) U.S. Cl. CPC C12Q 1/6886; C12Q 1/6881; C12Q 1/6851; C12Q 2600/158; G16B 20/00; G16B 25/10; C12Q 1/686

(57) ABSTRACT

The present disclosure is related to a developing method of candidate probes and a using method thereof. Specifically, the candidate probes are capable binding specific genes and further identifying a cell type of a tissue. Briefly, the developing method comprises the steps of: (a) using a chip to generate gene expressions of normal samples with known organ; (b) using a processing module to compare the gene expressions of the normal samples; and (c) developing candidate probes based on the previous comparing results. The using method comprises the steps of: (a′) using the previous candidate probes to detect the relative gene expression in a test sample with an unknown cell type; (b′) using a processing module to analysis the score of the test sample; and (c′) further predict the cell type of the test sample. Moreover, the present disclosure further provides a system used to conduct the above method, and the system comprises a detecting chip including an array with the candidate probes and a processing module.

The present application claims priority to U.S. Provisional Application Ser. No. 62/533,145, filed on Jul. 17, 2017, and PCT Application Serial No. PCT/CN2018/095,805, filed on Jul. 16, 2018, which are hereby incorporated by reference in their entirety.

### FIELD

The present disclosure relates to a method and a system for identifying a cell type, and more particularly to a method and a system for identifying whether a cell type is a normal/benign cell, a primary tumor cell or a metastatic tumor cell.

### BACKGROUND

Cancer has become the leading cause of deaths worldwide and has taken away millions of human lives every year during the past decades. (Ferlay J et al 2015). Treatment of cancers often involves costly, lengthy and painful processes. New methods of treatment such as target therapies and immuno-therapies are being promoted while cancer drug development is still strictly regulated by the governments of many countries. The anatomical pathological diagnosis is a subjective and traditional process which involves microscopic inspection of the biopsy slides. The interpretation on the morphology of the biopsies made by a pathologist is based on the pathologist's knowledge and experiences for the specific type of cancer. (Connolly J L et al, 2003) This process is considered the gold standard for cancer diagnosis as there has not been any superior technology available since it was firstly introduced around a century ago.

Due to the nature of a subjective process, it is not surprising that discrepancies may exist in certain cases when a biopsy slide is inspected by different pathologists. Systematic investigations on the accuracies of cancer diagnosis by anatomic pathology have uncovered significant discrepancy/error rates present in various medical institutes worldwide. (Nguyen et al 2004, Raab et al 2005, Elmore J G et al 2015, Singh H et al, 2007, Khazai L et al 2015, Mehrad M et al. 2015) For example, Raab et al reported 1% to 43% of error frequency in cancer diagnosis with anatomic pathology after reviewing more than a dozen of research articles published from 1984 to 2005. (Raab et al 2005) Having had 115 pathologists reviewing 60 cases of breast cancer biopsy slides, Elmore et al presented a 75.3% of concordance (i.e. 25% of discrepancies) with the previous reference diagnosis. (Elmore J G et al 2015) Nguyen et al found that 44% of the patients with adenocarcinomas of the prostate were changed for the Gleanson score by at least 1 point after second review on their pathological results by genitourinary oncologists. Some of the changes in diagnosis led to changes in treatments. (Nguyen et al 2004).

To reduce errors, the best solution as recommended by numerous medical institutes including The American Society of Clinical Pathologists is to have the biopsy slides reviewed by more than one pathologist. (John E. et al 2000, Nakhleh R E et al 2016, Middleton L P et al 2014, Leong A S et al 2006) Efforts in amending the procedure of surgical pathology also contributed to reducing diagnosis errors. (Nakhleh R E 2008, Nakhleh et al 2016) Application of immune-histochemical staining of selected marker proteins to the biopsy specimens facilitates cancer diagnosis to identify specific subtypes of a cancer. Despite tremendous efforts have been made to reduce the error rates caused at the surgical pathology, the ultimate solution to enhance the accuracy of cancer diagnosis would be to develop an objective diagnosis system which analyzed the specimen from an aspect other than morphology.

It is desirable to develop a method and a system to accurately and efficiently diagnosis whether a cell is a normal cell/benign tumor cell, a primary tumor cell or a metastatic tumor cell.

### SUMMARY

The present disclosure provides a gene-based prediction method with potential application in cancer diagnosis by taking advantage of the tissue-specific gene expression profiles. Also, the present disclosure demonstrates that a normal human tissue from each of the thirty anatomic sites exhibits a specific expression profile of the candidate genes in Table 1. The result was validated with a large scale meta-analysis on nearly eight hundred arrays coming from 61 different research groups and the accuracy of the validation reached 99.2%. Further, the result demonstrates that loss of normal tissue-specific expression profiles was found in those cells which had been transformed into a malignant tumor. Hence, the mathematical relationship (stoichiometry) of the relative expression levels of the candidate genes must be well maintained to ensure normal functioning and morphology of the tissue while the relationship becomes lost when the tissue turned cancerous.

By analysing meta-data and a number of clinical specimens from liver, the present disclosure demonstrates that the loss of stoichiometry in the expression levels of the marker genes may be a general phenomenon present in cancers. By taking both the clinical data and the computed scores into consideration, it was observed that the degree of deviation from a normal expression profile correlates with the extent of malignancies of a cancer (i.e. the degree of similarity is inversely correlated to the extent of cancer malignancies). Moreover, the present disclosure shows that a cancer can be characterized by using a multi-gene signature, which includes one or more genes in Table 1.

The present disclosure further provides a method for developing a plurality of candidate probes to identify a normal cell in a mammalian subject. The method includes the following steps: Step (a): using a detecting chip to generate a plurality of gene expression obtained from a standard sample of a subject either having or not having a selected disease, disorder or genetic pathology, and the standard sample is diagnosed with a normal cell of a known tissue; Step (b): using a processing module to compare the plurality of gene expressions to generate a comparison result; and Step (c): based on the comparison result, developing an array containing the plurality of candidate probes, wherein the plurality of candidate probes can bind a plurality of polynucleotide sequences selected from any one of SEQ ID No.1 to 652 or from any fragment of SEQ ID No.1 to 652. The detecting chip is connected (e.g., electrically or wirelessly) to the processing module.

In one embodiment, the number of candidate probes is about 200. In a preferred embodiment, the number of candidate probes is about 100. In a more preferred embodiment, the number of candidate probes is about 50-60. In the most preferred embodiment, the number of candidate probes is about 25-35.

In one embodiment, the standard sample includes blood, blood plasma, serum, urine, tissue, cells, organs, seminal fluids or any combination thereof.

In one embodiment, the selected disease, disorder or genetic disorder includes hematologic malignancies or solid tumors.

In one embodiment, the length of the candidate probes is about 15 nucleotides.

In one embodiment, the step (b) in the method for developing a plurality of candidate probes to identify a normal cell in a mammalian subject does not include: comparing the plurality of gene expressions for the standard sample with an abnormal sample of a subject diagnosed with a selected disease, disorder, genetic disorder or any combination thereof.

In one embodiment, the array in the step (c) of the method for developing a plurality of candidate probes to identify a normal cell in a mammalian subject is developed by applying the following: Pearson's correlation, Spearman's rank correlation, Kendall, k-means, Mahalanobis distance, Hamming distance, Levenshtein distance, Euclidean distances or any combination thereof.

In one embodiment, the step (c) in the method for developing a plurality of candidate probes to identify a normal cell in a mammalian subject further includes a step (c1): analyzing a correlation factor between an expression of a selected sequence of the plurality of the selected probes and an expression of the plurality of polynucleotide sequences selected from any one of SEQ ID No.1 to 652 or from any fragment of SEQ ID No.1 to 652. In further one embodiment, the correlation factor includes binding affinity.

The present disclosure also provides a method for characterizing the cell type of a tissue in a mammalian subject. The characterized method includes the following steps: Step (a′): using a detection chip containing the plurality of candidate probes mentioned previously to analyse the expression level of a test sample array obtained from a subject either having or not having a selected disease, disorder, genetic disorder, and the plurality of candidate probes can bind the plurality of polynucleotide sequence selected from any one of SEQ ID No.1 to 652 or from any fragment of SEQ ID No.1 to 652; Step (b′): using a processing module to calculate a score (e.g., a CM score) for the test sample based on the expression level of the array; and Step (c′): using the processing module to predict the cell type for the test sample based on the score (e.g., the CM score).

In one embodiment, the score used to predict the cell type for the test sample is a similarity or dissimilarity degree.

In one embodiment, the cell type of the test sample is characterized as a normal cell or a benign tumor cell when the CM score of the test sample is about >0.8.

In one embodiment, the cell type of the test sample is characterized as a primary tumor cell when the CM score of the test sample is about 0.8-0.3.

In one embodiment, the cell type of the test sample is characterized as a metastatic tumor cell when the CM score of the test sample is about <0.3.

In one embodiment, the cell type of the test sample is characterized as a normal cell or a benign tumor cell when the similarity degree of the test sample is about >80%. The cell type of the test sample is characterized as a primary tumor cell when the similarity degree of the test sample is about 30-80%. The cell type of the test sample is characterized as a metastatic tumor cell when the similarity degree of the test sample is about <30%. It is worth to know that the two subjects in comparison is identical when the similarity degree is 100%.

In one embodiment, the cell type of the test sample is characterized as a normal cell or a benign tumor cell when the dissimilarity degree of the test sample is about <20%. The cell type of the test sample is characterized as a primary tumor cell when the dissimilarity degree of the test sample is about 20-70%. The cell type of the test sample is characterized as a metastatic tumor cell when the dissimilarity degree of the test sample is about >70%. It is worth to know that the two subjects in comparison is identical when the dissimilarity degree is 0%.

In one embodiment, the test sample includes blood, blood plasma, serum, urine, tissue, cells, organs, seminal fluids or any combination thereof.

In one embodiment, the score in the step (b′) in the method for characterizing a cell type in a mammalian subject is generated by applying the following: Pearson's correlation coefficient, Spearman's rank correlation coefficient, Kendall, Mahalanobis distance, Euclidean distances or any combination thereof.

Furthermore, the present disclosure provides a system for characterizing the cell type of a tissue in a mammalian subject, and the system includes a detecting chip and a processing module. The processing module electrically connects to the detecting chip. The detecting chip contains a plurality of candidate probes that can bind a plurality of polynucleotide sequence selected from any one of SEQ ID No.1 to 652 or from any fragment of SEQ ID No.1 to 652. Furthermore, the detecting chip detects the expression level of a test sample array obtained from a subject having a selected disease, disorder, genetic disorder, and the processing module further calculates a CM score of the test sample based on the expression level of the array and then predicts the cell type of the test sample based on the CM score thereof.

In one embodiment, the number of the plurality of candidate probes in the system is about 200. In a preferred embodiment, the number of the plurality of candidate probes in the system is about 100. In a more preferred embodiment, the number of the plurality of candidate probes in the system is about 50-60. In a most preferred embodiment, the number of the plurality of candidate probes in the system is about 25-35.

In one embodiment, the test sample in the system includes blood, blood plasma, serum, urine, tissue, cells, organs, seminal fluids or any combination thereof.

In one embodiment, a length of the candidate probes in the system is at least 15 nucleotides.

Those and other aspects of the present disclosure may be further clarified by the following descriptions and drawings of preferred embodiments. Although there may be changes or modifications therein, they would not betray the spirit and scope of the novel ideas disclosed in the present disclosure.

### BRIEF DESCRIPTION OF THE DRAWINGS

One or more embodiments are illustrated by way of examples, and not by limitation, in the figures of the accompanying drawings, wherein elements having the same reference numeral designations represent like elements throughout. It should be understood that the present disclosure is not limited to the preferred embodiments shown. The data in the figures and examples are shown as mean±standard deviation (SD), determined by the paired t-test. Significant differences are shown as follows: *: P<0.05; **: P<0.01.

FIG. 1 discloses the example candidate genes resulted in complete tissue classification using standard two-way hierarchical clustering analysis. The columns indicate the tissue origins of the samples and the rows indicate the signature genes. The dendrogram shown on top of the heat map indicates the clustering of 30 tissues.

FIG. 2 discloses candidate genes of the present disclosure differentiating cancer from normal in multiple datasets. The averaged cancer malignancy scores (hereinafter the “CM scores”) of normal samples or tumors were computed for each dataset shown along the x axis. The source organ of the datasets are denoted below the GEO accession number. The open squares (designated N in the upper right corner) indicate the normal samples while the closed circles (designated T) the tumor samples. The means and error bars are shown as grey lines.

FIG. 3 discloses the distribution of CM scores by individual normal or cancer samples from selected datasets. The GEO accession number of the dataset was marked on top of the corresponding panel. The y axis indicates the CM score, and x axis indicates the category of the sample being normal (open square) or tumor (closed circle). The numerical values alone a grey line of a group of data points indicate the mean value of CM scores of the designated group. P-value was computed based on the one-tailed t-test and was shown as asterix (e.g. **** indicates p<0.0001).

FIGS. 4A and 4B show the results of the benign tumors or the near-benign cancers with the CM score analyses. FIG. 4A was from GSE33630 which consists of normal thyroid, papillary thyroid cancer (i.e., PTC) and anaplastic thyroid cancer (i.e., ATC). FIG. 4B showed the dataset GSE13319 which contained samples from myometrium (representing normal tissue of uterus, in red asterisk) and leiomyoma (representing a benign tumor from uterus, in open diamond).

The drawings are only schematic and are non-limiting. Any reference signs in the claims shall not be construed as limiting the scope. Like reference symbols in the various drawings indicate like elements

### DETAILED DESCRIPTION

Unless defined otherwise, all technical and scientific terms used herein have the same meanings as commonly understood by one of skill in the art to which this disclosure belongs. It will be further understood that terms; such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

### Definition

Unless clearly specified herein, meanings of the articles “a,” “an,” and “said” all include the plural form of “more than one.” Therefore, for example, when the term “a component” is used, it includes multiple said components and equivalents known to those of common knowledge in said field.

The term “about” and “around,” as used herein, when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “cancer” and “tumor” as used herein are both defined as a disease characterized by the rapid and uncontrolled growth of aberrant cells. Therefore, the terms of “cancer” and “tumor” are interchangeable. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

In the context of the present invention, the following abbreviations for the commonly occurring “nucleic acid bases” or “nucleotides” are used, “A” refers to adenosine, “C” refers to cytosine, “G” refers to guanosine, “T” refers to thymidine, and “U” refers to uridine.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

The term “candidate probe” and “selected probe” as used herein are both defined as the artificial probes generated by the present disclosure and capable of binding to the genes in Table 1. Therefore, the terms of “candidate probe” and “selected probe” are interchangeable.

TABLE 1
“Genes used as probes for identification”

| Gene sym | SEQID | Gene Title |
|---|---|---|
| FLJ14106 | 1 | Homo sapiens hypothetical protein FLJ14106, |
| | | mRNA″ |
| CSH2 | 2 | Homo sapiens chorionic somatomammotropin |
| | | hormone 2, transcript variant 1, mRNA″ |
| HLA-DRB6 | 3 | Homo sapiens major histocompatibility complex, class |
| | | II, DR beta 6 (pseudogene), non-coding RNA″ |
| WFDC10B | 4 | Homo sapiens WAP four-disulfide core domain 10B, |
| | | transcript variant 1, mRNA″ |
| EXOSC6 | 5 | Homo sapiens exosome component 6, mRNA″ |
| ZNF804A | 6 | Homo sapiens zinc finger protein 804A, mRNA″ |
| PCIF1 | 7 | Homo sapiens PDX1 C-terminal inhibiting factor 1, |
| | | mRNA″ |
| TCEAL2 | 8 | Homo sapiens transcription elongation factor A like 2, |
| | | mRNA″ |
| MS4A1 | 9 | Homo sapiens membrane spanning 4-domains A1, |
| | | transcript variant 3, mRNA″ |
| HOXA9 | 10 | Homo sapiens homeobox A9, mRNA″ |
| TMEM132A | 11 | Homo sapiens transmembrane protein 132A, transcript |
| | | variant 1, mRNA″ |
| ZNF750 | 12 | Homo sapiens zinc finger protein 750, mRNA″ |
| MYL1 | 13 | Homo sapiens myosin light chain 1, transcript variant |
| | | 1f, mRNA″ |
| GPR88 | 14 | Homo sapiens G protein-coupled receptor 88, mRNA″ |
| DNER | 15 | Homo sapiens delta/notch like EGF repeat containing, |
| | | mRNA″ |
| FRY | 16 | Homo sapiens FRY microtubule binding protein, |
| | | mRNA″ |
| SPEF2 | 17 | Homo sapiens sperm flagellar 2, transcript variant 1, |
| | | mRNA″ |
| C16orf54 | 18 | Homo sapiens chromosome 16 open reading frame 54, |
| | | mRNA″ |
| CBARP | 19 | Homo sapiens CACN beta subunit associated |
| | | regulatory protein, mRNA″ |
| PMAIP1 | 20 | Homo sapiens phorbol-12-myristate-13-acetate- |
| | | induced protein 1, mRNA″ |
| PAGR1 | 21 | Homo sapiens PAXIP1 associated glutamate rich |
| | | protein 1, mRNA″ |
| LIX1 | 22 | Homo sapiens limb and CNS expressed 1, mRNA″ |
| CA13 | 23 | Homo sapiens carbonic anhydrase 13, mRNA″ |
| TMPRSS11B | 24 | Homo sapiens transmembrane serine protease 11B, |
| | | mRNA″ |
| CNFN | 25 | Homo sapiens cornifelin, mRNA″ |
| ABRA | 26 | Homo sapiens actin binding Rho activating protein, |
| | | mRNA″ |
| JCHAIN | 27 | Homo sapiens joining chain of multimeric IgA and |
| | | IgM, mRNA″ |
| ZNF791 | 28 | Homo sapiens zinc finger protein 791, mRNA″ |
| ANO1 | 29 | Homo sapiens anoctamin 1, transcript variant 1, |
| | | mRNA″ |
| TMEM144 | 30 | Homo sapiens transmembrane protein 144, mRNA″ |
| NEFH | 31 | Homo sapiens neurofilament heavy, mRNA″ |
| VXN | 32 | Homo sapiens vexin, mRNA″ |
| CRCT1 | 33 | Homo sapiens cysteine rich C-terminal 1, mRNA″ |
| MIR155HG | 34 | Homo sapiens MIR155 host gene, long non-coding |
| | | RNA″ |
| CREG2 | 35 | Homo sapiens cellular repressor of E1A stimulated |
| | | genes 2, mRNA″ |
| TUBB2B | 36 | Homo sapiens tubulin beta 2B class IIb, mRNA″ |
| SLC17A6 | 37 | Homo sapiens solute carrier family 17 member 6, |
| | | mRNA″ |
| PERP | 38 | Homo sapiens PERP, TP53 apoptosis effector, |
| | | mRNA″ |
| TXLNB | 39 | Homo sapiens taxilin beta, mRNA″ |
| LINC01105 | 40 | Homo sapiens long intergenic non-protein coding |
| | | RNA 1105, long non-coding RNA″ |
| PCDH9 | 41 | Homo sapiens protocadherin 9, transcript variant 2, |
| | | mRNA″ |
| GPAT4 | 42 | Homo sapiens glycerol-3-phosphate acyltransferase 4, |
| | | mRNA″ |
| OLIG1 | 43 | Homo sapiens oligodendrocyte transcription factor 1, |
| | | mRNA″ |
| MTERF4 | 44 | Homo sapiens mitochondrial transcription termination |
| | | factor 4, transcript variant 1, mRNA″ |
| LINC00632 | 45 | Homo sapiens long intergenic non-protein coding |
| | | RNA 632, transcript variant 1, long non-coding RNA″ |
| ZC3H12D | 46 | Homo sapiens zinc finger CCCH-type containing 12D, |
| | | mRNA″ |
| C11orf87 | 47 | Homo sapiens chromosome 11 open reading frame 87, |
| | | mRNA″ |
| ASB5 | 48 | Homo sapiens ankyrin repeat and SOCS box |
| | | containing 5, mRNA″ |
| LINC00944 | 49 | Homo sapiens long intergenic non-protein coding |
| | | RNA 944, long non-coding RNA″ |
| RNF144A-AS1 | 50 | Homo sapiens RNF144A antisense RNA 1, long non- |
| | | coding RNA″ |
| UBE2Z | 51 | Homo sapiens ubiquitin conjugating enzyme E2 Z, |
| | | mRNA″ |
| UBAC2-AS1 | 52 | Homo sapiens UBAC2 antisense RNA 1, transcript |
| | | variant 1, long non-coding RNA″ |
| LOC100506965 | 53 | Homo sapiens hypothetical LOC100506965, |
| | | miscRNA″ |
| BRICD5 | 54 | Homo sapiens BRICHOS domain containing 5, |
| | | mRNA″ |
| DSTNP2 | 55 | Homo sapiens destrin, actin depolymerizing factor |
| | | pseudogene 2, non-coding RNA″ |
| MAMDC2-AS1 | 56 | Homo sapiens MAMDC2 antisense RNA 1, long non- |
| | | coding RNA″ |
| MYOZ2 | 57 | Homo sapiens myozenin 2, mRNA″ |
| LRRC2 | 58 | Homo sapiens leucine rich repeat containing 2, |
| | | mRNA″ |
| APOOL | 59 | Homo sapiens apolipoprotein O like, mRNA″ |
| HCN1 | 60 | Homo sapiens hyperpolarization activated cyclic |
| | | nucleotide gated potassium channel 1, mRNA″ |
| TCIM | 61 | Homo sapiens transcriptional and immune response |
| | | regulator, mRNA″ |
| FDCSP | 62 | Homo sapiens follicular dendritic cell secreted protein, |
| | | mRNA″ |
| MTUS2-AS1 | 63 | Homo sapiens MTUS2 antisense RNA 1, long non- |
| | | coding RNA″ |
| XIST | 64 | Homo sapiens X inactive specific transcript (non- |
| | | protein coding), long non-coding RNA″ |
| GATAD1 | 65 | Homo sapiens GATA zinc finger domain containing |
| | | 1, transcript variant 1, mRNA″ |
| C15orf48 | 66 | Homo sapiens chromosome 15 open reading frame 48, |
| | | transcript variant 2, mRNA″ |
| CSGALNACT2 | 67 | Homo sapiens chondroitin sulfate N- |
| | | acetylgalactosaminyltransferase 2, transcript variant 1, |
| | | mRNA″ |
| SOX2-OT | 68 | Homo sapiens SOX2 overlapping transcript, transcript |
| | | variant 4, long non-coding RNA″ |
| C16orf58 | 69 | Homo sapiens chromosome 16 open reading frame 58, |
| | | mRNA″ |
| ACKR4 | 70 | Homo sapiens atypical chemokine receptor 4, |
| | | transcript variant 2, mRNA″ |
| P2RY12 | 71 | Homo sapiens purinergic receptor P2Y12, transcript |
| | | variant 1, mRNA″ |
| LOC101927513 | 72 | Homo sapiens uncharacterized LOC101927513, |
| | | ncRNA″ |
| LOC100507642 | 73 | Homo sapiens uncharacterized LOC100507642, |
| | | transcript variant 1, long non-coding RNA″ |
| LINC00844 | 74 | Homo sapiens long intergenic non-protein coding |
| | | RNA 844, long non-coding RNA″ |
| AMER2 | 75 | Homo sapiens APC membrane recruitment protein 2, |
| | | transcript variant 1, mRNA″ |
| FAM83C-AS1 | 76 | Homo sapiens FAM83C antisense RNA 1, long non- |
| | | coding RNA″ |
| LINC01215 | 77 | Homo sapiens long intergenic non-protein coding |
| | | RNA 1215, transcript variant 1, long non-coding |
| | | RNA″ |
| ANKRD44-IT1 | 78 | Homo sapiens ANKRD44 intronic transcript 1, long |
| | | non-coding RNA″ |
| MIR133A1HG | 79 | Homo sapiens MIR133A1 host gene, long non-coding |
| | | RNA″ |
| LINC01770 | 80 | Homo sapiens long intergenic non-protein coding |
| | | RNA 1770, transcript variant 1, long non-coding |
| | | RNA″ |
| AGR3 | 81 | Homo sapiens anterior gradient 3, protein disulphide |
| | | isomerase family member, mRNA″ |
| DIRAS2 | 82 | Homo sapiens DIRAS family GTPase 2, mRNA″ |
| PCDH10 | 83 | Homo sapiens protocadherin 10, transcript variant 2, |
| | | mRNA″ |
| NEK5 | 84 | Homo sapiens NIMA related kinase 5, mRNA″ |
| PPP3R2 | 85 | Homo sapiens protein phosphatase 3 regulatory |
| | | subunit B, beta, mRNA″ |
| LOC105373660 | 86 | Homo sapiens uncharacterized LOC105373660, |
| | | transcript variant X4, ncRNA″ |
| LOC101930370 | 87 | Homo sapiens uncharacterized LOC101930370, |
| | | transcript variant X1, ncRNA″ |
| TRAT1 | 88 | Homo sapiens T cell receptor associated |
| | | transmembrane adaptor 1, transcript variant 1, |
| | | mRNA″ |
| SPX | 89 | Homo sapiens spexin hormone, transcript variant 1, |
| | | mRNA″ |
| TMTC2 | 90 | Homo sapiens transmembrane and tetratricopeptide |
| | | repeat containing 2, transcript variant 1, mRNA″ |
| VGLL3 | 91 | Homo sapiens vestigial like family member 3, |
| | | transcript variant 1, mRNA″ |
| COL14A1 | 92 | Homo sapiens collagen type XIV alpha 1 chain, |
| | | mRNA″ |
| LOC285556 | 93 | Homo sapiens uncharacterized LOC285556, transcript |
| | | variant X1, mRNA″ |
| ZNF467 | 94 | Homo sapiens zinc finger protein 467, transcript |
| | | variant 1, mRNA″ |
| LMOD2 | 95 | Homo sapiens leiomodin 2, mRNA″ |
| TCEAL7 | 96 | Homo sapiens transcription elongation factor A like 7, |
| | | transcript variant 1, mRNA″ |
| PRPF40A | 97 | Homo sapiens pre-mRNA processing factor 40 |
| | | homolog A, transcript variant 1, mRNA″ |
| ZFAS1 | 98 | Homo sapiens ZNFX1 antisense RNA 1, transcript |
| | | variant 1, long non-coding RNA″ |
| FAM192A | 99 | Homo sapiens family with sequence similarity 192 |
| | | member A, transcript variant 20, mRNA″ |
| LINC00461 | 100 | Homo sapiens long intergenic non-protein coding |
| | | RNA 461, transcript variant 3, long non-coding RNA″ |
| S100A12 | 101 | Homo sapiens S100 calcium binding protein A12, |
| | | mRNA″ |
| MRPS28 | 102 | Homo sapiens mitochondrial ribosomal protein S28, |
| | | mRNA″ |
| ITK | 103 | Homo sapiens IL2 inducible T cell kinase, mRNA″ |
| LHX2 | 104 | Homo sapiens LIM homeobox 2, mRNA″ |
| PELO | 105 | Homo sapiens pelota mRNA surveillance and |
| | | ribosome rescue factor, mRNA″ |
| CDK5R1 | 106 | Homo sapiens cyclin dependent kinase 5 regulatory |
| | | subunit 1, mRNA″ |
| CPLX1 | 107 | Homo sapiens complexin 1, mRNA″ |
| CDC40 | 108 | Homo sapiens cell division cycle 40, mRNA″ |
| PANX1 | 109 | Homo sapiens pannexin 1, mRNA″ |
| CLIC3 | 110 | Homo sapiens chloride intracellular channel 3, |
| | | mRNA″ |
| KLHL41 | 111 | Homo sapiens kelch like family member 41, mRNA″ |
| CDR1 | 112 | Homo sapiens cerebellar degeneration related protein |
| | | 1, mRNA″ |
| MB | 113 | Homo sapiens myoglobin, transcript variant 1, |
| | | mRNA″ |
| S100A2 | 114 | Homo sapiens S100 calcium binding protein A2, |
| | | mRNA″ |
| S100P | 115 | Homo sapiens S100 calcium binding protein P, |
| | | mRNA″ |
| RIMS3 | 116 | Homo sapiens regulating synaptic membrane |
| | | exocytosis 3, mRNA″ |
| PCP4 | 117 | Homo sapiens Purkinje cell protein 4, mRNA″ |
| CFL1 | 118 | Homo sapiens cofilin 1, mRNA″ |
| RBP4 | 119 | Homo sapiens retinol binding protein 4, transcript |
| | | variant 1, mRNA″ |
| MLLT11 | 120 | Homo sapiens MLLT11, transcription factor 7 |
| | | cofactor, mRNA″ |
| CELA2B | 121 | Homo sapiens chymotrypsin like elastase family |
| | | member 2B, mRNA″ |
| CSTA | 122 | Homo sapiens cystatin A, mRNA″ |
| NNMT | 123 | Homo sapiens nicotinamide N-methyltransferase, |
| | | mRNA″ |
| DKK4 | 124 | Homo sapiens dickkopf WNT signaling pathway |
| | | inhibitor 4, mRNA″ |
| KRT7 | 125 | Homo sapiens keratin 7, mRNA″ |
| MEOX2 | 126 | Homo sapiens mesenchyme homeobox 2, mRNA″ |
| CLCA3 | 127 | Homo sapiens chloride channel, calcium activated, |
| | | family member 3, mRNA″ |
| CD96 | 128 | Homo sapiens CD96 molecule, transcript variant 2, |
| | | mRNA″ |
| SMR3B | 129 | Homo sapiens submaxillary gland androgen regulated |
| | | protein 3B, mRNA″ |
| PNLIPRP2 | 130 | Homo sapiens pancreatic lipase related protein 2 |
| | | (gene/pseudogene), transcript variant 1, coding, |
| | | mRNA″ |
| MTF1 | 131 | Homo sapiens metal regulatory transcription factor 1, |
| | | mRNA″ |
| S100B | 132 | Homo sapiens S100 calcium binding protein B, |
| | | mRNA″ |
| MYH1 | 133 | Homo sapiens myosin heavy chain 1, mRNA″ |
| GREB1 | 134 | Homo sapiens growth regulating estrogen receptor |
| | | binding 1, transcript variant a, mRNA″ |
| HDDC2 | 135 | Homo sapiens HD domain containing 2, mRNA″ |
| PSD3 | 136 | Homo sapiens pleckstrin and Sec7 domain containing |
| | | 3, transcript variant 1, mRNA″ |
| KRT6B | 137 | Homo sapiens keratin 6B, mRNA″ |
| KRT6A | 138 | Homo sapiens keratin 6A, mRNA″ |
| FUT9 | 139 | Homo sapiens fucosyltransferase 9, mRNA″ |
| CEP68 | 140 | Homo sapiens centrosomal protein 68, transcript |
| | | variant 1, mRNA″ |
| PNMA2 | 141 | Homo sapiens PNMA family member 2, mRNA″ |
| POU2AF1 | 142 | Homo sapiens POU class 2 associating factor 1, |
| | | mRNA″ |
| FUT7) | 143 | Homo sapiens fucosyltransferase 7, mRNA″ |
| REG1B | 144 | Homo sapiens regenerating family member 1 beta, |
| | | mRNA″ |
| ASCL1 | 145 | Homo sapiens achaete-scute family bHLH |
| | | transcription factor 1, mRNA″ |
| COL6A3 | 146 | Homo sapiens collagen type VI alpha 3 chain, |
| | | transcript variant 1, mRNA″ |
| SERPINB3 | 147 | Homo sapiens serpin family B member 3, mRNA″ |
| GJB2 | 148 | Homo sapiens gap junction protein beta 2, mRNA″ |
| CYTIP | 149 | Homo sapiens cytohesin 1 interacting protein, mRNA″ |
| ST18 | 150 | Homo sapiens ST18, C2H2C-type zinc finger, |
| | | transcript variant 1, mRNA″ |
| CADPS | 151 | Homo sapiens calcium dependent secretion activator, |
| | | transcript variant 1, mRNA″ |
| AKAP12 | 152 | Homo sapiens A-kinase anchoring protein 12, |
| | | transcript variant 1, mRNA″ |
| CA3 | 153 | Homo sapiens carbonic anhydrase 3, mRNA″ |
| LACTB2 | 154 | Homo sapiens lactamase beta 2, mRNA″ |
| AGR2 | 155 | Homo sapiens anterior gradient 2, protein disulphide |
| | | isomerase family member, mRNA″ |
| PAX9 | 156 | Homo sapiens paired box 9, mRNA″ |
| GABBR2 | 157 | Homo sapiens gamma-aminobutyric acid type B |
| | | receptor subunit 2, mRNA″ |
| MPZL2 | 158 | Homo sapiens myelin protein zero like 2, transcript |
| | | variant 1, mRNA″ |
| AVIL | 159 | Homo sapiens advillin, mRNA″ |
| PCOLCE2 | 160 | Homo sapiens procollagen C-endopeptidase enhancer |
| | | 2, mRNA″ |
| WIF1 | 161 | Homo sapiens WNT inhibitory factor 1, mRNA″ |
| VAMP8 | 162 | Homo sapiens vesicle associated membrane protein 8, |
| | | mRNA″ |
| (ZNF770 | 163 | Homo sapiens zinc finger protein 770, mRNA″ |
| COMMD2 | 164 | Homo sapiens COMM domain containing 2, transcript |
| | | variant 1, mRNA″ |
| SCG2 | 165 | Homo sapiens secretogranin II, mRNA″ |
| FEZ1 | 166 | Homo sapiens fasciculation and elongation protein |
| | | zeta 1, transcript variant 1, mRNA″ |
| SYNGR3 | 167 | Homo sapiens synaptogyrin 3, mRNA″ |
| NAP1L3 | 168 | Homo sapiens nucleosome assembly protein 1 like 3, |
| | | mRNA″ |
| OLFM4 | 169 | Homo sapiens olfactomedin 4, mRNA″ |
| AQP3 | 170 | Homo sapiens aquaporin 3 (Gill blood group), |
| | | transcript variant 1, mRNA″ |
| KIF5C | 171 | Homo sapiens kinesin family member 5C, transcript |
| | | variant 1, mRNA″ |
| MYL9 | 172 | Homo sapiens myosin light chain 9, transcript variant |
| | | 1, mRNA″ |
| FOXG1 | 173 | Homo sapiens forkhead box G1, mRNA″ |
| CSRP3 | 174 | Homo sapiens cysteine and glycine rich protein 3, |
| | | mRNA″ |
| NEFL | 175 | Homo sapiens neurofilament light, mRNA″ |
| ZFYVE9 | 176 | Homo sapiens zinc finger FYVE-type containing 9, |
| | | transcript variant 3, mRNA″ |
| SHANK2 | 177 | Homo sapiens SH3 and multiple ankyrin repeat |
| | | domains 2, transcript variant 1, mRNA″ |
| GATA6 | 178 | Homo sapiens GATA binding protein 6, mRNA″ |
| HS3ST3B1 | 179 | Homo sapiens heparan sulfate-glucosamine 3- |
| | | sulfotransferase 3B1, transcript variant 1, mRNA″ |
| CALB1 | 180 | Homo sapiens calbindin 1, mRNA″ |
| POU3F3 | 181 | Homo sapiens POU class 3 homeobox 3, mRNA″ |
| CDH1 | 182 | Homo sapiens cadherin 1, transcript variant 1, |
| | | mRNA″ |
| OGN | 183 | Homo sapiens osteoglycin, transcript variant 3, |
| | | mRNA″ |
| HDAC6 | 184 | Homo sapiens histone deacetylase 6, transcript variant |
| | | 5, mRNA″ |
| DHRS7 | 185 | Homo sapiens dehydrogenase/reductase 7, transcript |
| | | variant 1, mRNA″ |
| PIAS2 | 186 | Homo sapiens protein inhibitor of activated STAT 2, |
| | | transcript variant beta, mRNA″ |
| FRRS1L | 187 | Homo sapiens ferric chelate reductase 1 like, mRNA″ |
| SCRG1 | 188 | Homo sapiens stimulator of chondrogenesis 1, |
| | | transcript variant 2, mRNA″ |
| GDF15 | 189 | Homo sapiens growth differentiation factor 15, |
| | | mRNA″ |
| GZMB | 190 | Homo sapiens granzyme B, transcript variant 1, |
| | | mRNA″ |
| CNTN2 | 191 | Homo sapiens contactin 2, transcript variant 1, |
| | | mRNA″ |
| CLCA2 | 192 | Homo sapiens chloride channel accessory 2, mRNA″ |
| LCP2 | 193 | Homo sapiens lymphocyte cytosolic protein 2, |
| | | mRNA″ |
| WSB1 | 194 | Homo sapiens WD repeat and SOCS box containing 1, |
| | | transcript variant 1, mRNA″ |
| ZIC2 | 195 | Homo sapiens Zic family member 2, mRNA″ |
| TNRC6A | 196 | Homo sapiens trinucleotide repeat containing 6A, |
| | | transcript variant 1, mRNA″ |
| ATP8B1 | 197 | Homo sapiens ATPase phospholipid transporting 8B1, |
| | | mRNA″ |
| GPR37 | 198 | Homo sapiens G protein-coupled receptor 37, mRNA″ |
| COQ2 | 199 | Homo sapiens coenzyme Q2, polyprenyltransferase, |
| | | transcript variant 1, mRNA″ |
| APOA2 | 200 | Homo sapiens apolipoprotein A2, mRNA″ |
| ENO2 | 201 | Homo sapiens enolase 2, mRNA″ |
| CST1 | 202 | Homo sapiens cystatin SN, mRNA″ |
| TNNC2 | 203 | Homo sapiens troponin C2, fast skeletal type, mRNA″ |
| ELAVL3 | 204 | Homo sapiens ELAV like RNA binding protein 3, |
| | | transcript variant 1, mRNA″ |
| HLA-DQA1 | 205 | Homo sapiens major histocompatibility complex, class |
| | | II, DQ alpha 1, mRNA″ |
| ITGA9 | 206 | Homo sapiens integrin subunit alpha 9, mRNA″ |
| DES | 207 | Homo sapiens desmin, mRNA″ |
| RGS1 | 208 | Homo sapiens regulator of G protein signaling 1, |
| | | mRNA″ |
| FLG | 209 | Homo sapiens filaggrin, mRNA″ |
| LUM | 210 | Homo sapiens lumican, mRNA″ |
| VSNL1 | 211 | Homo sapiens visinin like 1, mRNA″ |
| CD52 | 212 | Homo sapiens CD52 molecule, mRNA″ |
| ZIC1 | 213 | Homo sapiens Zic family member 1, mRNA″ |
| SPRR1B | 214 | Homo sapiens small proline rich protein 1B, mRNA″ |
| S100A9 | 215 | Homo sapiens S100 calcium binding protein A9, |
| | | mRNA″ |
| S100A7 | 216 | Homo sapiens S100 calcium binding protein A7, |
| | | mRNA″ |
| NID1 | 217 | Homo sapiens nidogen 1, mRNA″ |
| COL6A2 | 218 | Homo sapiens collagen type VI alpha 2 chain, |
| | | transcript variant 2C2, mRNA″ |
| EREG | 219 | Homo sapiens epiregulin, mRNA″ |
| DSG3 | 220 | Homo sapiens desmoglein 3, mRNA″ |
| PRM1 | 221 | Homo sapiens protamine 1, mRNA″ |
| KRT13 | 222 | Homo sapiens keratin 13, transcript variant 2, mRNA″ |
| KRT19 | 223 | Homo sapiens keratin 19, mRNA″ |
| TNP1 | 224 | Homo sapiens transition protein 1, mRNA″ |
| TEAD3 | 225 | Homo sapiens TEA domain transcription factor 3, |
| | | mRNA″ |
| CXCL2 | 226 | Homo sapiens C-X-C motif chemokine ligand 2, |
| | | mRNA″ |
| PITX1 | 227 | Homo sapiens paired like homeodomain 1, mRNA″ |
| ADGRB3 | 228 | Homo sapiens adhesion G protein-coupled receptor |
| | | B3, mRNA″ |
| TAC1 | 229 | Homo sapiens tachykinin precursor 1, transcript |
| | | variant beta, mRNA″ |
| TACSTD2 | 230 | Homo sapiens tumor associated calcium signal |
| | | transducer 2, mRNA″ |
| PPP1R3A | 231 | Homo sapiens protein phosphatase 1 regulatory |
| | | subunit 3A, mRNA″ |
| PTX3 | 232 | Homo sapiens pentraxin 3, mRNA″ |
| FABP4 | 233 | Homo sapiens fatty acid binding protein 4, mRNA″ |
| SFRP4 | 234 | Homo sapiens secreted frizzled related protein 4, |
| | | mRNA″ |
| PCK1 | 235 | Homo sapiens phosphoenolpyruvate carboxykinase 1, |
| | | mRNA″ |
| AMBP | 236 | Homo sapiens alpha-1-microglobulin/bikunin |
| | | precursor, mRNA″ |
| SLC6A1 | 237 | Homo sapiens solute carrier family 6 member 1, |
| | | transcript variant 1, mRNA″ |
| SCGB2A1 | 238 | Homo sapiens secretoglobin family 2A member 1, |
| | | mRNA″ |
| PRKCB | 239 | Homo sapiens protein kinase C beta, transcript variant |
| | | 2, mRNA″ |
| EMP1 | 240 | Homo sapiens epithelial membrane protein 1, mRNA″ |
| TNNC1 | 241 | Homo sapiens troponin C1, slow skeletal and cardiac |
| | | type, mRNA″ |
| BTG1 | 242 | Homo sapiens BTG anti-proliferation factor 1, |
| | | mRNA″ |
| KRT15 | 243 | Homo sapiens keratin 15, mRNA″ |
| EPCAM | 244 | Homo sapiens epithelial cell adhesion molecule, |
| | | mRNA″ |
| CHGB | 245 | Homo sapiens chromogranin B, mRNA″ |
| CD69 | 246 | Homo sapiens CD69 molecule, mRNA″ |
| PIGR | 247 | Homo sapiens polymeric immunoglobulin receptor, |
| | | mRNA″ |
| PPBP | 248 | Homo sapiens pro-platelet basic protein, mRNA″ |
| DPT | 249 | Homo sapiens dermatopontin, mRNA″ |
| REG3A | 250 | Homo sapiens regenerating family member 3 alpha, |
| | | transcript variant 1, mRNA″ |
| S100A8 | 251 | Homo sapiens S100 calcium binding protein A8, |
| | | transcript variant 4, mRNA″ |
| NKX2-2 | 252 | Homo sapiens NK2 homeobox 2, mRNA″ |
| THRSP | 253 | Homo sapiens thyroid hormone responsive, mRNA″ |
| H3F3A | 254 | Homo sapiens H3 histone family member 3A, mRNA″ |
| PCDH8 | 255 | Homo sapiens protocadherin 8, transcript variant 1, |
| | | mRNA″ |
| FABP1 | 256 | Homo sapiens fatty acid binding protein 1, mRNA″ |
| SOX2 | 257 | Homo sapiens SRY-box 2, mRNA″ |
| MSMB | 258 | Homo sapiens microseminoprotein beta, transcript |
| | | variant PSP94, mRNA″ |
| CSH1 | 259 | Homo sapiens chorionic somatomammotropin |
| | | hormone 1, mRNA″ |
| STRN | 260 | Homo sapiens striatin, mRNA″ |
| EEF1A2 | 261 | Homo sapiens eukaryotic translation elongation factor |
| | | 1 alpha 2, mRNA″ |
| CKM | 262 | Homo sapiens creatine kinase, M-type, mRNA″ |
| GCG | 263 | Homo sapiens glucagon, mRNA″ |
| CEL | 264 | Homo sapiens carboxyl ester lipase, mRNA″ |
| CXCL5 | 265 | Homo sapiens C-X-C motif chemokine ligand 5, |
| | | mRNA″ |
| COL15A1 | 266 | Homo sapiens collagen type XV alpha 1 chain, |
| | | mRNA″ |
| YWHAB | 267 | Homo sapiens tyrosine 3-monooxygenase/tryptophan |
| | | 5-monooxygenase activation protein beta, transcript |
| | | variant 1, mRNA″ |
| SCGB2A2 | 268 | Homo sapiens secretoglobin family 2A member 2, |
| | | mRNA″ |
| SH3GL2 | 269 | Homo sapiens SH3 domain containing GRB2 like 2, |
| | | endophilin A1, mRNA″ |
| SPINK1 | 270 | Homo sapiens serine peptidase inhibitor, Kazal type 1, |
| | | transcript variant 2, mRNA″ |
| SERPINB4 | 271 | Homo sapiens serpin family B member 4, transcript |
| | | variant 1, mRNA″ |
| HTN1 | 272 | Homo sapiens histatin 1, mRNA″ |
| CPA1 | 273 | Homo sapiens carboxypeptidase A1, mRNA″ |
| FCAR | 274 | Homo sapiens Fc fragment of IgA receptor, transcript |
| | | variant 1, mRNA″ |
| CFAP47 | 275 | Homo sapiens cilia and flagella associated protein 47, |
| | | transcript variant 1, mRNA″ |
| APOBEC1 | 276 | Homo sapiens apolipoprotein B mRNA editing |
| | | enzyme catalytic subunit 1, transcript variant 2, |
| | | mRNA″ |
| CRTAM | 277 | Homo sapiens cytotoxic and regulatory T cell |
| | | molecule, transcript variant 2, mRNA″ |
| CKS2 | 278 | Homo sapiens CDC28 protein kinase regulatory |
| | | subunit 2, mRNA″ |
| DSG1 | 279 | Homo sapiens desmoglein 1, mRNA″ |
| TMEFF2 | 280 | Homo sapiens transmembrane protein with EGF like |
| | | and two follistatin like domains 2, transcript variant 2, |
| | | mRNA″ |
| THBS1 | 281 | Homo sapiens thrombospondin 1, mRNA″ |
| SEPT11 | 282 | Homo sapiens septin 11, transcript variant 1, mRNA″ |
| SERPINB13 | 283 | Homo sapiens serpin family B member 13, transcript |
| | | variant 1, mRNA″ |
| EED | 284 | Homo sapiens embryonic ectoderm development, |
| | | transcript variant 3, mRNA″ |
| LGI1 | 285 | Homo sapiens leucine rich glioma inactivated 1, |
| | | transcript variant 2, mRNA″ |
| ADAM32 | 286 | Homo sapiens ADAM metallopeptidase domain 32, |
| | | transcript variant 2, mRNA″ |
| DCN | 287 | Homo sapiens decorin, transcript variant A1, mRNA″ |
| CPE | 288 | Homo sapiens carboxypeptidase E, mRNA″ |
| LSAMP | 289 | Homo sapiens limbic system associated membrane |
| | | protein, transcript variant 1, mRNA″ |
| FABP7 | 290 | Homo sapiens fatty acid binding protein 7, transcript |
| | | variant 1, mRNA″ |
| CSHL1 | 291 | Homo sapiens chorionic somatomammotropin |
| | | hormone like 1, transcript variant 3, mRNA″ |
| SNAP25 | 292 | Homo sapiens synaptosome associated protein 25, |
| | | transcript variant 1, mRNA″ |
| PLN | 293 | Homo sapiens phospholamban, mRNA″ |
| INHBA | 294 | Homo sapiens inhibin beta A subunit, mRNA″ |
| PTN | 295 | Homo sapiens pleiotrophin, transcript variant 1, |
| | | mRNA″ |
| MNDA | 296 | Homo sapiens myeloid cell nuclear differentiation |
| | | antigen, mRNA″ |
| PMP2 | 297 | Homo sapiens peripheral myelin protein 2, transcript |
| | | variant 1, mRNA″ |
| AHSG | 298 | Homo sapiens alpha 2-HS glycoprotein, transcript |
| | | variant 2, mRNA″ |
| AQP4 | 299 | Homo sapiens aquaporin 4, transcript variant 1, |
| | | mRNA″ |
| CAMK2B | 300 | Homo sapiens calcium/calmodulin dependent protein |
| | | kinase II beta, transcript variant 1, mRNA″ |
| AZGP1 | 301 | Homo sapiens alpha-2-glycoprotein 1, zinc-binding, |
| | | mRNA″ |
| ADIPOQ | 302 | Homo sapiens adiponectin, C1Q and collagen domain |
| | | containing, transcript variant 1, mRNA″ |
| IGLL5 | 303 | Homo sapiens immunoglobulin lambda like |
| | | polypeptide 5, transcript variant 1, mRNA″ |
| BCAT1 | 304 | Homo sapiens branched chain amino acid |
| | | transaminase 1, transcript variant 2, mRNA″ |
| SUFU | 305 | Homo sapiens SUFU negative regulator of hedgehog |
| | | signaling, transcript variant 2, mRNA″ |
| CPEB3 | 306 | Homo sapiens cytoplasmic polyadenylation element |
| | | binding protein 3, transcript variant 2, mRNA″ |
| FGB | 307 | Homo sapiens fibrinogen beta chain, transcript variant |
| | | 2, mRNA″ |
| TUT7 | 308 | Homo sapiens terminal uridylyl transferase 7, |
| | | transcript variant 2, mRNA″ |
| RPH3AL | 309 | Homo sapiens rabphilin 3A like (without C2 |
| | | domains), transcript variant 2, mRNA″ |
| NCOR1 | 310 | Homo sapiens nuclear receptor corepressor 1, |
| | | transcript variant 2, mRNA″ |
| GREM1 | 311 | Homo sapiens gremlin 1, DAN family BMP |
| | | antagonist, transcript variant 3, mRNA″ |
| ENO3 | 312 | Homo sapiens enolase 3 (ENO3), transcript variant 3, |
| | | mRNA″ |
| MATR3 | 313 | Homo sapiens matrin 3, transcript variant 3, mRNA″ |
| DCLK1 | 314 | Homo sapiens doublecortin like kinase 1, transcript |
| | | variant 2, mRNA″ |
| LOC100505841 | 315 | Homo sapiens zinc finger protein 474-like, mRNA″ |
| CAMTA1 | 316 | Homo sapiens calmodulin binding transcription |
| | | activator 1, transcript variant 2, mRNA″ |
| RUNX1T1 | 317 | Homo sapiens RUNX1 translocation partner 1, |
| | | transcript variant 5, mRNA″ |
| SEPT4 | 318 | Homo sapiens septin 4, transcript variant 4, mRNA″ |
| LIPF | 319 | Homo sapiens lipase F, gastric type, transcript variant |
| | | 3, mRNA″ |
| MSANTD3- | 320 | Homo sapiens MSANTD3-TMEFF1 readthrough, |
| TMEFF1 | | mRNA″ |
| DCTN5 | 321 | Homo sapiens dynactin subunit 5, transcript variant 2, |
| | | mRNA″ |
| LTF | 322 | Homo sapiens lactotransferrin, transcript variant 2, |
| | | mRNA″ |
| STMN2 | 323 | Homo sapiens stathmin 2, transcript variant 1, |
| | | mRNA″ |
| PHACTR3 | 324 | Homo sapiens phosphatase and actin regulator 3, |
| | | transcript variant 4, mRNA″ |
| CTSS | 325 | Homo sapiens cathepsin S, transcript variant 2, |
| | | mRNA″ |
| INTS7 | 326 | Homo sapiens integrator complex subunit 7, transcript |
| | | variant 4, mRNA″ |
| SPRR1A | 327 | Homo sapiens small proline rich protein 1A, transcript |
| | | variant 1, mRNA″ |
| WDR27 | 328 | Homo sapiens WD repeat domain 27, transcript |
| | | variant 2, mRNA″ |
| ANKS1B | 329 | Homo sapiens ankyrin repeat and sterile alpha motif |
| | | domain containing 1B, transcript variant 4, mRNA″ |
| PRPS1 | 330 | Homo sapiens phosphoribosyl pyrophosphate |
| | | synthetase 1, transcript variant 2, mRNA″ |
| SORT1 | 331 | Homo sapiens sortilin 1, transcript variant 2, mRNA″ |
| EHF | 332 | Homo sapiens ETS homologous factor, transcript |
| | | variant 3, mRNA″ |
| RFX4 | 333 | Homo sapiens regulatory factor X4, transcript variant |
| | | 4, mRNA″ |
| PTPRZ1 | 334 | Homo sapiens protein tyrosine phosphatase, receptor |
| | | type Z1, transcript variant 2, mRNA″ |
| SNAP91 | 335 | Homo sapiens synaptosome associated protein 91, |
| | | transcript variant 3, mRNA″ |
| RTN1 | 336 | Homo sapiens reticulon 1, transcript variant 4, |
| | | mRNA″ |
| SLC24A2 | 337 | Homo sapiens solute carrier family 24 member 2, |
| | | transcript variant 2, mRNA″ |
| GNG2 | 338 | Homo sapiens G protein subunit gamma 2, transcript |
| | | variant 2, mRNA″ |
| GFPT1 | 339 | Homo sapiens glutamine--fructose-6-phosphate |
| | | transaminase 1, transcript variant 1, mRNA″ |
| KRTDAP | 340 | Homo sapiens keratinocyte differentiation associated |
| | | protein, transcript variant 2, mRNA″ |
| TRDN | 341 | Homo sapiens triadin, transcript variant 2, mRNA″ |
| CLPS | 342 | Homo sapiens colipase, transcript variant 2, mRNA″ |
| SLC1A2 | 343 | Homo sapiens solute carrier family 1 member 2, |
| | | transcript variant 2, mRNA″ |
| CHL1 | 344 | Homo sapiens cell adhesion molecule L1 like, |
| | | transcript variant 2, mRNA″ |
| AKR1C3 | 345 | Homo sapiens aldo-keto reductase family 1 member |
| | | C3, transcript variant 2, mRNA″ |
| CYB5D2 | 346 | Homo sapiens cytochrome b5 domain containing 2, |
| | | transcript variant 2, mRNA″ |
| CNTN1 | 347 | Homo sapiens contactin 1, transcript variant 3, |
| | | mRNA″ |
| TDRP | 348 | Homo sapiens testis development related protein, |
| | | transcript variant 2, mRNA″ |
| SAMSN1 | 349 | Homo sapiens SAM domain, SH3 domain and nuclear |
| | | localization signals 1, transcript variant 2, mRNA″ |
| CACNA1G | 350 | Homo sapiens calcium voltage-gated channel subunit |
| | | alpha1 G, transcript variant 16, mRNA″ |
| MEGF10 | 351 | Homo sapiens multiple EGF like domains 10, |
| | | transcript variant 2, mRNA″ |
| ENC1 | 352 | Homo sapiens ectodermal-neural cortex 1, transcript |
| | | variant 2, mRNA″ |
| CCT4 | 353 | Homo sapiens chaperonin containing TCP1 subunit 4, |
| | | transcript variant 2, mRNA″ |
| PEX5L | 354 | Homo sapiens peroxisomal biogenesis factor 5 like, |
| | | transcript variant 2, mRNA″ |
| TTN | 355 | Homo sapiens titin, transcript variant N2BA, mRNA″ |
| DNAJC6 | 356 | Homo sapiens DnaJ heat shock protein family (Hsp40) |
| | | member C6, transcript variant 1, mRNA″ |
| CLCN4 | 357 | Homo sapiens chloride voltage-gated channel 4, |
| | | transcript variant 2, mRNA″ |
| DDX11 | 358 | Homo sapiens DEAD/H-box helicase 11, transcript |
| | | variant 4, mRNA″ |
| GPM6A | 359 | Homo sapiens glycoprotein M6A, transcript variant 4, |
| | | mRNA″ |
| INSL3 | 360 | Homo sapiens insulin like 3, transcript variant 1, |
| | | mRNA″ |
| PTPRC | 361 | Homo sapiens protein tyrosine phosphatase, receptor |
| | | type C, transcript variant 5, mRNA″ |
| PKIB | 362 | Homo sapiens cAMP-dependent protein kinase |
| | | inhibitor beta, transcript variant 4, mRNA″ |
| KCNJ16 | 363 | Homo sapiens potassium voltage-gated channel |
| | | subfamily J member 16, transcript variant 4, mRNA″ |
| NRM | 364 | Homo sapiens nurim, transcript variant 2, mRNA″ |
| TFPI2 | 365 | Homo sapiens tissue factor pathway inhibitor 2, |
| | | transcript variant 2, mRNA″ |
| JPH3 | 366 | Homo sapiens junctophilin 3, transcript variant 2, |
| | | mRNA″ |
| PNLDC1 | 367 | Homo sapiens PARN like, ribonuclease domain |
| | | containing 1, transcript variant 1, mRNA″ |
| GANAB | 368 | Homo sapiens glucosidase II alpha subunit, transcript |
| | | variant 4, mRNA″ |
| MOBP | 369 | Homo sapiens myelin-associated oligodendrocyte |
| | | basic protein, transcript variant 1, mRNA″ |
| TAGAP | 370 | Homo sapiens T cell activation RhoGTPase activating |
| | | protein, transcript variant 4, mRNA″ |
| CSMD2 | 371 | Homo sapiens CUB and Sushi multiple domains 2, |
| | | transcript variant 1, mRNA″ |
| PPFIA2 | 372 | Homo sapiens PTPRF interacting protein alpha 2, |
| | | transcript variant 2, mRNA″ |
| OLFM1 | 373 | Homo sapiens olfactomedin 1, transcript variant 4, |
| | | mRNA″ |
| STMN4 | 374 | Homo sapiens stathmin 4, transcript variant 2, |
| | | mRNA″ |
| PRM2 | 375 | Homo sapiens protamine 2, transcript variant 2, |
| | | mRNA″ |
| KLF5 | 376 | Homo sapiens Kruppel like factor 5, transcript variant |
| | | 2, mRNA″ |
| CTNND2 | 377 | Homo sapiens catenin delta 2, transcript variant 2, |
| | | mRNA″ |
| GMIP | 378 | Homo sapiens GEM interacting protein, transcript |
| | | variant 2, mRNA″ |
| SMARCA2 | 379 | Homo sapiens SWI/SNF related, matrix associated, |
| | | actin dependent regulator of chromatin, subfamily a, |
| | | member 2, transcript variant 3, mRNA″ |
| CRYAB | 380 | Homo sapiens crystallin alpha B, transcript variant 2, |
| | | mRNA″ |
| TPTE | 381 | Homo sapiens transmembrane phosphatase with tensin |
| | | homology, transcript variant 4, mRNA″ |
| CD24 | 382 | Homo sapiens CD24 molecule, transcript variant 2, |
| | | mRNA″ |
| UGT2B4 | 383 | Homo sapiens UDP glucuronosyltransferase family 2 |
| | | member B4, transcript variant 2, mRNA″ |
| MFAP5 | 384 | Homo sapiens microfibril associated protein 5, |
| | | transcript variant 2, mRNA″ |
| SYDE1 | 385 | Homo sapiens synapse defective Rho GTPase |
| | | homolog 1, transcript variant 2, mRNA″ |
| QKI | 386 | Homo sapiens QKI, KH domain containing RNA |
| | | binding, transcript variant 5, mRNA″ |
| CCR7 | 387 | Homo sapiens C-C motif chemokine receptor 7, |
| | | transcript variant 2, mRNA″ |
| ANLN | 388 | Homo sapiens anillin actin binding protein, transcript |
| | | variant 2, mRNA″ |
| MYT1L | 389 | Homo sapiens myelin transcription factor 1 like, |
| | | transcript variant 1, mRNA″ |
| PRUNE1 | 390 | Homo sapiens prune exopolyphosphatase 1, transcript |
| | | variant 2, mRNA″ |
| PRSS2 | 391 | Homo sapiens serine protease 2, transcript variant 1, |
| | | mRNA″ |
| ARMC7 | 392 | Homo sapiens armadillo repeat containing 7, transcript |
| | | variant 2, mRNA″ |
| LMOD3 | 393 | Homo sapiens leiomodin 3, transcript variant 2, |
| | | mRNA″ |
| STXBP6 | 394 | Homo sapiens syntaxin binding protein 6, transcript |
| | | variant 2, mRNA″ |
| HNRNPUL1 | 395 | Homo sapiens heterogeneous nuclear |
| | | ribonucleoprotein U like 1, transcript variant 5, |
| | | mRNA″ |
| RNF217 | 396 | Homo sapiens ring finger protein 217, transcript |
| | | variant 1, mRNA″ |
| FILIP1 | 397 | Homo sapiens filamin A interacting protein 1, |
| | | transcript variant 1, mRNA″ |
| CRISP3 | 398 | Homo sapiens cysteine rich secretory protein 3, |
| | | transcript variant 2, mRNA″ |
| RGS7 | 399 | Homo sapiens regulator of G protein signaling 7, |
| | | transcript variant 2, mRNA″ |
| ACTA1 | 400 | Homo sapiens actin, alpha 1, skeletal muscle, mRNA″ |
| SST | 401 | Homo sapiens somatostatin, mRNA″ |
| SPOCK3 | 402 | Homo sapiens SPARC (osteonectin), cwcv and kazal |
| | | like domains proteoglycan 3, transcript variant 1, |
| | | mRNA″ |
| SCN2A | 403 | Homo sapiens sodium voltage-gated channel alpha |
| | | subunit 2, transcript variant 2, mRNA″ |
| ZNF557 | 404 | Homo sapiens zinc finger protein 557, transcript |
| | | variant 2, mRNA″ |
| ANKRD7 | 405 | Homo sapiens ankyrin repeat domain 7, transcript |
| | | variant 1, mRNA″ |
| ONECUT3 | 406 | Homo sapiens one cut homeobox 3, mRNA″ |
| SNTN | 407 | Homo sapiens sentan, cilia apical structure protein, |
| | | transcript variant 2, mRNA″ |
| DEFA1B | 408 | Homo sapiens defensin alpha 1B, transcript variant 2, |
| | | mRNA″ |
| SPRR3 | 409 | Homo sapiens small proline rich protein 3, transcript |
| | | variant 2, mRNA″ |
| MYH2 | 410 | Homo sapiens myosin heavy chain 2, transcript |
| | | variant 2, mRNA″ |
| RAPGEF4 | 411 | Homo sapiens Rap guanine nucleotide exchange |
| | | factor 4, transcript variant 2, mRNA″ |
| PNMA8A | 412 | Homo sapiens PNMA family member 8A, transcript |
| | | variant 2, mRNA″ |
| NEFM | 413 | Homo sapiens neurofilament medium, transcript |
| | | variant 2, mRNA″ |
| PRH2 | 414 | Homo sapiens proline rich protein HaeIII subfamily 2, |
| | | mRNA″ |
| NAA16 | 415 | Homo sapiens N(alpha)-acetyltransferase 16, NatA |
| | | auxiliary subunit, transcript variant 3, mRNA″ |
| SLC8A1 | 416 | Homo sapiens solute carrier family 8 member A1, |
| | | transcript variant B, mRNA″ |
| CLIC5 | 417 | Homo sapiens chloride intracellular channel 5, |
| | | transcript variant 1, mRNA″ |
| BCL2A1 | 418 | Homo sapiens BCL2 related protein A1, transcript |
| | | variant 2, mRNA″ |
| SERPINI1 | 419 | Homo sapiens serpin family I member 1, transcript |
| | | variant 2, mRNA″ |
| NRGN | 420 | Homo sapiens neurogranin, transcript variant 2, |
| | | mRNA″ |
| DIAPH1 | 421 | Homo sapiens diaphanous related formin 1, transcript |
| | | variant 2, mRNA″ |
| SALL1 | 422 | Homo sapiens spalt like transcription factor 1, |
| | | transcript variant 2, mRNA″ |
| SYNPR | 423 | Homo sapiens synaptoporin, transcript variant 1, |
| | | mRNA″ |
| PLEKHB1 | 424 | Homo sapiens pleckstrin homology domain containing |
| | | B1, transcript variant 3, mRNA″ |
| GAP43 | 425 | Homo sapiens growth associated protein 43, transcript |
| | | variant 1, mRNA″ |
| TRIM2 | 426 | Homo sapiens tripartite motif containing 2, transcript |
| | | variant 2, mRNA″ |
| KLC1 | 427 | Homo sapiens kinesin light chain 1, transcript variant |
| | | 3, mRNA″ |
| GJB6 | 428 | Homo sapiens gap junction protein beta 6, transcript |
| | | variant 1, mRNA″ |
| NDRG4 | 429 | Homo sapiens NDRG family member 4, transcript |
| | | variant 2, mRNA″ |
| HMGB2 | 430 | Homo sapiens high mobility group box 2, transcript |
| | | variant 2, mRNA″ |
| PLAC8 | 431 | Homo sapiens placenta specific 8, transcript variant 3, |
| | | mRNA″ |
| CDC2 | 432 | Homo sapiens cell division cycle 2, G1 to S and G2 to |
| | | M, transcript variant 3, mRNA″ |
| MAP4 | 433 | Homo sapiens microtubule associated protein 4, |
| | | transcript variant 4, mRNA″ |
| SLC12A5 | 434 | Homo sapiens solute carrier family 12 member 5, |
| | | transcript variant 1, mRNA″ |
| ZSCAN31 | 435 | Homo sapiens zinc finger and SCAN domain |
| | | containing 31, transcript variant 3, mRNA″ |
| SYT1 | 436 | Homo sapiens synaptotagmin 1, transcript variant 2, |
| | | mRNA″ |
| MYOT | 437 | Homo sapiens myotilin, transcript variant 2, mRNA″ |
| POSTN | 438 | Homo sapiens periostin, transcript variant 2, mRNA″ |
| LRRFIP1 | 439 | Homo sapiens LRR binding FLII interacting protein 1, |
| | | transcript variant 1, mRNA″ |
| SERPINB2 | 440 | Homo sapiens serpin family B member 2, transcript |
| | | variant 1, mRNA″ |
| MUC7 | 441 | Homo sapiens mucin 7, secreted, transcript variant 1, |
| | | mRNA″ |
| CPT1B | 442 | Homo sapiens carnitine palmitoyltransferase 1B, |
| | | transcript variant 5, mRNA″ |
| C12orf75 | 443 | Homo sapiens chromosome 12 open reading frame 75, |
| | | mRNA″ |
| ADAMDEC1 | 444 | Homo sapiens ADAM like decysin 1, transcript |
| | | variant 2, mRNA″ |
| TPM2 | 445 | Homo sapiens tropomyosin 2 (beta), transcript variant |
| | | 3, mRNA″ |
| MMP1 | 446 | Homo sapiens matrix metallopeptidase 1, transcript |
| | | variant 2, mRNA″ |
| PEG3 | 447 | Homo sapiens paternally expressed 3, transcript |
| | | variant 2, mRNA″ |
| MPZL1 | 448 | Homo sapiens myelin protein zero like 1, transcript |
| | | variant 3, mRNA″ |
| ETNPPL | 449 | Homo sapiens ethanolamine-phosphate phospholyase, |
| | | transcript variant 2, mRNA″ |
| SLC39A11 | 450 | Homo sapiens solute carrier family 39 member 11, |
| | | transcript variant 1, mRNA″ |
| SCEL | 451 | Homo sapiens sciellin, transcript variant 3, mRNA″ |
| MAFF | 452 | Homo sapiens MAF bZIP transcription factor F, |
| | | transcript variant 3, mRNA″ |
| WWC1 | 453 | Homo sapiens WW and C2 domain containing 1, |
| | | transcript variant 1, mRNA″ |
| TF | 454 | Homo sapiens transferrin, transcript variant 1, mRNA″ |
| NEB | 455 | Homo sapiens nebulin, transcript variant 1, mRNA″ |
| SCG3 | 456 | Homo sapiens secretogranin III, transcript variant 2, |
| | | mRNA″ |
| CALM1 | 457 | Homo sapiens calmodulin 1 (phosphorylase kinase, |
| | | delta), transcript variant 2, mRNA″ |
| CADM2 | 458 | Homo sapiens cell adhesion molecule 2, transcript |
| | | variant 1, mRNA″ |
| ATRAID | 459 | Homo sapiens all-trans retinoic acid induced |
| | | differentiation factor, transcript variant 3, mRNA″ |
| FAM122C | 460 | Homo sapiens family with sequence similarity 122C, |
| | | transcript variant 1, mRNA″ |
| SIGLEC10 | 461 | Homo sapiens sialic acid binding Ig like lectin 10, |
| | | transcript variant 2, mRNA″ |
| ELAVL2 | 462 | Homo sapiens ELAV like RNA binding protein 2, |
| | | transcript variant 2, mRNA″ |
| FAAP20 | 463 | Homo sapiens Fanconi anemia core complex |
| | | associated protein 20, transcript variant 1, mRNA″ |
| CSRNP3 | 464 | Homo sapiens cysteine and serine rich nuclear protein |
| | | 3, transcript variant 1, mRNA″ |
| NEXN | 465 | Homo sapiens nexilin F-actin binding protein, |
| | | transcript variant 2, mRNA″ |
| MYD88 | 466 | Homo sapiens myeloid differentiation primary |
| | | response 88, transcript variant 5, mRNA″ |
| BANP | 467 | Homo sapiens BTG3 associated nuclear protein, |
| | | transcript variant 3, mRNA″ |
| GBP5 | 468 | Homo sapiens guanylate binding protein 5, transcript |
| | | variant 2, mRNA″ |
| XIRP2 | 469 | Homo sapiens xin actin binding repeat containing 2, |
| | | transcript variant 2, mRNA″ |
| PRR4 | 470 | Homo sapiens proline rich 4, transcript variant 1, |
| | | mRNA″ |
| GFAP | 471 | Homo sapiens glial fibrillary acidic protein, transcript |
| | | variant 2, mRNA″ |
| SLAIN1 | 472 | Homo sapiens SLAIN motif family member 1, |
| | | transcript variant 1, mRNA″ |
| PDLIM3 | 473 | Homo sapiens PDZ and LIM domain 3, transcript |
| | | variant 2, mRNA″ |
| HMGCS1 | 474 | Homo sapiens 3-hydroxy-3-methylglutaryl-CoA |
| | | synthase 1, transcript variant 1, mRNA″ |
| CRISP2 | 475 | Homo sapiens cysteine rich secretory protein 2, |
| | | transcript variant 2, mRNA″ |
| SZRD1 | 476 | Homo sapiens SUZ RNA binding domain containing |
| | | 1, transcript variant 1, mRNA″ |
| GBA3 | 477 | Homo sapiens glucosylceramidase beta 3 |
| | | (gene/pseudogene), transcript variant 2, coding, |
| | | mRNA″ |
| DST | 478 | Homo sapiens dystonin, transcript variant 2, mRNA″ |
| DNM3 | 479 | Homo sapiens dynamin 3, transcript variant 2, |
| | | mRNA″ |
| ACTN2 | 480 | Homo sapiens actinin alpha 2, transcript variant 1, |
| | | mRNA″ |
| MAPK3 | 481 | Homo sapiens mitogen-activated protein kinase 3, |
| | | transcript variant 2, mRNA″ |
| TIMM17B | 482 | Homo sapiens translocase of inner mitochondrial |
| | | membrane 17B, transcript variant 1, mRNA″ |
| ACSF3 | 483 | Homo sapiens acyl-CoA synthetase family member 3, |
| | | transcript variant 2, mRNA″ |
| OSR2 | 484 | Homo sapiens odd-skipped related transciption factor |
| | | 2, transcript variant 1, mRNA″ |
| SYNPO2L | 485 | Homo sapiens synaptopodin 2 like, transcript variant |
| | | 1, mRNA″ |
| IFT22 | 486 | Homo sapiens intraflagellar transport 22, transcript |
| | | variant 2, mRNA″ |
| CPN2 | 487 | Homo sapiens carboxypeptidase N subunit 2, |
| | | transcript variant 1, mRNA″ |
| NKAIN2 | 488 | Homo sapiens sodium/potassium transporting ATPase |
| | | interacting 2, transcript variant 1, mRNA″ |
| PRG4 | 489 | Homo sapiens proteoglycan 4, transcript variant B, |
| | | mRNA″ |
| EML4 | 490 | Homo sapiens echinoderm microtubule associated |
| | | protein like 4, transcript variant 2, mRNA″ |
| CLEC12B | 491 | Homo sapiens C-type lectin domain family 12 |
| | | member B, transcript variant 1, mRNA″ |
| UGT8 | 492 | Homo sapiens UDP glycosyltransferase 8, transcript |
| | | variant 1, mRNA″ |
| ZCWPW2 | 493 | Homo sapiens zinc finger CW-type and PWWP |
| | | domain containing 2, transcript variant 1, mRNA″ |
| PAK3 | 494 | Homo sapiens p21 (RAC1) activated kinase 3, |
| | | transcript variant 1, mRNA″ |
| SCG5 | 495 | Homo sapiens secretogranin V, transcript variant 1, |
| | | mRNA″ |
| NRXN1 | 496 | Homo sapiens neurexin 1, transcript variant alpha2, |
| | | mRNA″ |
| SCN1A | 497 | Homo sapiens sodium voltage-gated channel alpha |
| | | subunit 1, transcript variant 1, mRNA″ |
| ANK2 | 498 | Homo sapiens ankyrin 2, transcript variant 3, mRNA″ |
| RC3H2 | 499 | Homo sapiens ring finger and CCCH-type domains 2, |
| | | transcript variant 1, mRNA″ |
| | 500 | Homo sapiens CREB gene, exon Y″ |
| C8orf8 gene | 501 | Homo sapiens partial mRNA for hypothetical protein |
| | 502 | Homo sapiens IGH mRNA for immunoglobulin heavy |
| | | chain VHDJ region, partial cds, clone:H184″ |
| HBG2 | 503 | Homo sapiens hemoglobin subunit gamma 2, mRNA″ |
| PLA2G1B | 504 | Homo sapiens phospholipase A2 group IB, mRNA″ |
| SPP1 | 505 | Homo sapiens secreted phosphoprotein 1, transcript |
| | | variant 2, mRNA″ |
| KRT18 | 506 | Homo sapiens keratin 18, transcript variant 1, mRNA″ |
| COL1A2 | 507 | Homo sapiens collagen type I alpha 2 chain, mRNA″ |
| GATA3 | 508 | Homo sapiens GATA binding protein 3, transcript |
| | | variant 1, mRNA″ |
| HNRNPL | 509 | Homo sapiens heterogeneous nuclear |
| | | ribonucleoprotein L, transcript variant 2, mRNA″ |
| METTL2A | 510 | Homo sapiens methyltransferase like 2A, mRNA″ |
| STAR | 511 | Homo sapiens steroidogenic acute regulatory protein, |
| | | mRNA″ |
| STATH | 512 | Homo sapiens statherin, transcript variant 2, mRNA″ |
| VWA8 | 513 | Homo sapiens von Willebrand factor A domain |
| | | containing 8, transcript variant 2, mRNA″ |
| GAD1 | 514 | Homo sapiens glutamate decarboxylase 1, transcript |
| | | variant GAD67, mRNA″ |
| CLDN18 | 515 | Homo sapiens claudin 18, transcript variant 2, |
| | | mRNA″ |
| AKT1 | 516 | Homo sapiens AKT serine/threonine kinase 1, |
| | | transcript variant 3, mRNA″ |
| TPM1 | 517 | Homo sapiens tropomyosin 1, transcript variant |
| | | Tpm1.5, mRNA″ |
| DKK3 | 518 | Homo sapiens dickkopf WNT signaling pathway |
| | | inhibitor 3, transcript variant 3, mRNA″ |
| BAALC | 519 | Homo sapiens BAALC, MAP3K1 and KLF4 binding, |
| | | transcript variant 2, mRNA″ |
| ARPP21 | 520 | Homo sapiens cAMP regulated phosphoprotein 21, |
| | | transcript variant 3, mRNA″ |
| MBP | 521 | Homo sapiens myelin basic protein, transcript variant |
| | | 1, mRNA″ |
| KIAA0020 | 522 | Homo sapiens KIAA0020, transcript variant 1, |
| | | mRNA″ |
| KYNU | 523 | Homo sapiens kynureninase, transcript variant 2, |
| | | mRNA″ |
| DLK1 | 524 | Homo sapiens delta-like 1 homolog (Drosophila), |
| | | transcript variant 2, mRNA″ |
| C12orf37 | 525 | Homo sapiens chromosome 12 open reading frame 37, |
| | | mRNA″ |
| PART1 | 526 | Homo sapiens prostate androgen-regulated transcript |
| | | 1, mRNA″ |
| MAP2 | 527 | Homo sapiens microtubule associated protein 2, |
| | | transcript variant 5, mRNA″ |
| VTN | 528 | Homo sapiens vitronectin, mRNA″ |
| LOC643923 | 529 | Homo sapiens hypothetical protein LOC643923, |
| | | mRNA″ |
| COL3A1 | 530 | Homo sapiens collagen type III alpha 1 chain, mRNA″ |
| COL1A1 | 531 | Homo sapiens collagen type I alpha 1 chain, mRNA″ |
| ADORA1 | 532 | Homo sapiens adenosine A1 receptor, transcript |
| | | variant 1, mRNA″ |
| CTRB2 | 533 | Homo sapiens chymotrypsinogen B2, mRNA″ |
| KRT5 | 534 | Homo sapiens keratin 5, mRNA″ |
| GABRB2 | 535 | Homo sapiens gamma-aminobutyric acid type A |
| | | receptor beta2 subunit, transcript variant 2, mRNA″ |
| IL2 | 536 | Homo sapiens interleukin 2, mRNA″ |
| SLC12A1 | 537 | Homo sapiens solute carrier family 12 member 1, |
| | | transcript variant 1, mRNA″ |
| GRIA2 | 538 | Homo sapiens glutamate ionotropic receptor AMPA |
| | | type subunit 2, transcript variant 1, mRNA″ |
| FLG2 | 539 | Homo sapiens filaggrin family member 2, mRNA″ |
| TNNI3 | 540 | Homo sapiens troponin I3, cardiac type, mRNA″ |
| PITX2 | 541 | Homo sapiens paired like homeodomain 2, transcript |
| | | variant 3, mRNA″ |
| CYP11A1 | 542 | Homo sapiens cytochrome P450 family 11 subfamily |
| | | A member 1, transcript variant 1, mRNA″ |
| ECE2 | 543 | Homo sapiens endothelin converting enzyme 2, |
| | | transcript variant 2, mRNA″ |
| ACSM2A | 544 | Homo sapiens acyl-CoA synthetase medium-chain |
| | | family member 2A, transcript variant 3, mRNA″ |
| RHAG | 545 | Homo sapiens Rh associated glycoprotein, mRNA″ |
| CALN1 | 546 | Homo sapiens calneuron 1, transcript variant 2, |
| | | mRNA″ |
| CA2 | 547 | Homo sapiens carbonic anhydrase 2, transcript variant |
| | | 1, mRNA″ |
| GRIA3 | 548 | Homo sapiens glutamate ionotropic receptor AMPA |
| | | type subunit 3, transcript variant 2, mRNA″ |
| ORM1 | 549 | Homo sapiens orosomucoid 1, mRNA″ |
| LYZ | 550 | Homo sapiens lysozyme, mRNA″ |
| SLC3A1 | 551 | Homo sapiens solute carrier family 3 member 1, |
| | | mRNA″ |
| CD36 | 552 | Homo sapiens CD36 molecule, transcript variant 3, |
| | | mRNA″ |
| ABAT | 553 | Homo sapiens 4-aminobutyrate aminotransferase, |
| | | transcript variant 2, mRNA″ |
| GABRA1 | 554 | Homo sapiens gamma-aminobutyric acid type A |
| | | receptor alphal subunit, transcript variant 1, mRNA″ |
| GABRG2 | 555 | Homo sapiens gamma-aminobutyric acid type A |
| | | receptor gamma2 subunit, transcript variant 2, |
| | | mRNA″ |
| SERPINA1 | 556 | Homo sapiens serpin family A member 1, transcript |
| | | variant 1, mRNA″ |
| MYL2 | 557 | Homo sapiens myosin light chain 2, mRNA″ |
| GABRB1 | 558 | Homo sapiens gamma-aminobutyric acid type A |
| | | receptor betal subunit, mRNA″ |
| TECRL | 559 | Homo sapiens trans-2,3-enoyl-CoA reductase like, |
| | | mRNA″ |
| MTUS1 | 560 | Homo sapiens microtubule associated scaffold protein |
| | | 1, transcript variant 1, mRNA″ |
| KRT14 | 561 | Homo sapiens keratin 14, mRNA″ |
| NOS2 | 562 | Homo sapiens nitric oxide synthase 2, mRNA″ |
| ATP1A2 | 563 | Homo sapiens ATPase Na+/K+ transporting subunit |
| | | alpha 2, mRNA″ |
| IFNA2 | 564 | Homo sapiens interferon alpha 2, mRNA″ |
| ALDOB | 565 | Homo sapiens aldolase, fructose-bisphosphate B , |
| | | mRNA″ |
| ACAT1 | 566 | Homo sapiens acetyl-CoA acetyltransferase 1 , |
| | | mRNA″ |
| STXBP1 | 567 | Homo sapiens syntaxin binding protein 1, transcript |
| | | variant 2, mRNA″ |
| HTN3 | 568 | Homo sapiens histatin 3, mRNA″ |
| NHSL2 | 569 | Homo sapiens NHS like 2, mRNA″ |
| LRTM2 | 570 | Homo sapiens leucine rich repeats and transmembrane |
| | | domains 2, transcript variant 1, mRNA″ |
| GABRA5 | 571 | Homo sapiens gamma-aminobutyric acid type A |
| | | receptor alpha5 subunit, transcript variant 1, mRNA″ |
| RRM2 | 572 | Homo sapiens ribonucleotide reductase regulatory |
| | | subunit M2, transcript variant 2, mRNA″ |
| EVI2A | 573 | Homo sapiens ecotropic viral integration site 2A, |
| | | transcript variant 1, mRNA″ |
| MOG | 574 | Homo sapiens myelin oligodendrocyte glycoprotein, |
| | | transcript variant alpha3, mRNA″ |
| AMPD1 | 575 | Homo sapiens adenosine monophosphate deaminase |
| | | 1, transcript variant 1, mRNA″ |
| SAR1B | 576 | Homo sapiens secretion associated Ras related |
| | | GTPase 1B, transcript variant 1, mRNA″ |
| TFG | 577 | Homo sapiens TRK-fused gene, transcript variant 2, |
| | | mRNA″ |
| TTYH1 | 578 | Homo sapiens tweety family member 1, transcript |
| | | variant 2, mRNA″ |
| GC | 579 | Homo sapiens vitamin D binding protein (GC), |
| | | transcript variant 1, mRNA″ |
| CXCL8 | 580 | Homo sapiens C-X-C motif chemokine ligand 8 , |
| | | transcript variant 1, mRNA″ |
| ACSL6 | 581 | Homo sapiens acyl-CoA synthetase long chain family |
| | | member 6, transcript variant 2, mRNA″ |
| DLGAP1 | 582 | Homo sapiens DLG associated protein 1, transcript |
| | | variant 2, mRNA″ |
| NTRK3 | 583 | Homo sapiens neurotrophic receptor tyrosine kinase |
| | | 3, transcript variant 3, mRNA″ |
| MSMO1 | 584 | Homo sapiens methylsterol monooxygenase 1 , |
| | | transcript variant 2, mRNA″ |
| HPGD | 585 | Homo sapiens 15-hydroxyprostaglandin |
| | | dehydrogenase, transcript variant 1, mRNA″ |
| PDLIM5 | 586 | Homo sapiens PDZ and LIM domain 5, transcript |
| | | variant 2, mRNA″ |
| CLEC2D | 587 | Homo sapiens C-type lectin domain family 2 member |
| | | D, transcript variant 2, mRNA″ |
| G6PC | 588 | Homo sapiens glucose-6-phosphatase catalytic |
| | | subunit, transcript variant 1, mRNA″ |
| C6orf58 | 589 | Homo sapiens chromosome 6 open reading frame 58 , |
| | | mRNA″ |
| DNAJB14 | 590 | Homo sapiens DnaJ heat shock protein family (Hsp40) |
| | | member B14, transcript variant 1, mRNA″ |
| ADH1B | 591 | Homo sapiens alcohol dehydrogenase 1B (class I), |
| | | beta polypeptide, transcript variant 1, mRNA″ |
| DNM1 | 592 | Homo sapiens dynamin 1, transcript variant 2, |
| | | mRNA″ |
| DPP6 | 593 | Homo sapiens dipeptidyl peptidase like 6, transcript |
| | | variant 3, mRNA″ |
| NTRK2 | 594 | Homo sapiens neurotrophic receptor tyrosine kinase |
| | | 2, transcript variant b, mRNA″ |
| RUFY3 | 595 | Homo sapiens RUN and FYVE domain containing 3, |
| | | transcript variant 1, mRNA″ |
| GRIN2A | 596 | Homo sapiens glutamate ionotropic receptor NMDA |
| | | type subunit 2A, transcript variant 2, mRNA″ |
| GJA1 | 597 | Homo sapiens gap junction protein alpha 1, mRNA″ |
| GH1 | 598 | Homo sapiens growth hormone 1, transcript variant 1, |
| | | mRNA″ |
| MYH7 | 599 | Homo sapiens myosin heavy chain 7, mRNA″ |
| PLP1 | 600 | Homo sapiens proteolipid protein 1, transcript variant |
| | | 1, mRNA″ |
| AMY2A | 601 | Homo sapiens amylase, alpha 2A (pancreatic), |
| | | mRNA″ |
| ERMN | 602 | Homo sapiens ermin, transcript variant 1, mRNA″ |
| FGG | 603 | Homo sapiens fibrinogen gamma chain, transcript |
| | | variant gamma, mRNA″ |
| APOA1 | 604 | Homo sapiens apolipoprotein A1, transcript variant 1, |
| | | mRNA″ |
| FGA | 605 | Homo sapiens fibrinogen alpha chain, transcript |
| | | variant alpha-E, mRNA″ |
| GPM6B | 606 | Homo sapiens glycoprotein M6B, transcript variant 4, |
| | | mRNA″ |
| DSP | 607 | Homo sapiens desmoplakin, transcript variant 2, |
| | | mRNA″ |
| OPCML | 608 | Homo sapiens opioid binding protein/cell adhesion |
| | | molecule like, transcript variant 2, mRNA″ |
| ALOX5 | 609 | Homo sapiens arachidonate 5-lipoxygenase, transcript |
| | | variant 1, mRNA″ |
| APLP1 | 610 | Homo sapiens amyloid beta precursor like protein 1, |
| | | transcript variant 1, mRNA″ |
| PNLIP | 611 | Homo sapiens pancreatic lipase, mRNA″ |
| ALB | 612 | Homo sapiens albumin, mRNA″ |
| GABRA2 | 613 | Homo sapiens gamma-aminobutyric acid type A |
| | | receptor alpha2 subunit, transcript variant 1, mRNA″ |
| MGP | 614 | Homo sapiens matrix Gla protein, transcript variant 2, |
| | | mRNA″ |
| CXCR4 | 615 | Homo sapiens C-X-C motif chemokine receptor 4, |
| | | transcript variant 1, mRNA″ |
| RBFOX2 | 616 | Homo sapiens RNA binding fox-1 homolog 2, |
| | | transcript variant 1, mRNA″ |
| IGSF11 | 617 | Homo sapiens immunoglobulin superfamily member |
| | | 11, transcript variant 2, mRNA″ |
| IGFBP1 | 618 | Homo sapiens insulin like growth factor binding |
| | | protein 1, mRNA″ |
| KCNJ5 | 619 | Homo sapiens potassium voltage-gated channel |
| | | subfamily J member 5, transcript variant 1, mRNA″ |
| PAH | 620 | Homo sapiens phenylalanine hydroxylase, transcript |
| | | variant 1, mRNA″ |
| APOC3 | 621 | Homo sapiens apolipoprotein C3, mRNA″ |
| WT1 | 622 | Homo sapiens Wilms tumor 1, transcript variant A, |
| | | mRNA″ |
| | 623 | Homo sapiens CREB gene, exon Y″ |
| | 624 | Human mRNA upregulated during camptothecin- |
| | | induced apoptosis of U937 cells |
| | 625 | Homo sapiens unknown protein mRNA, partial cds″ |
| | 626 | Homo sapiens genomic DNA; cDNA |
| | | DKFZp586I1319 (from clone DKFZp586I1319) |
| | 627 | Homo sapiens clone IMAGE: 121662 mRNA sequence |
| | 628 | Homo sapiens genomic DNA; cDNA |
| | | DKFZp434F0728 (from clone DKFZp434F0728) |
| | 629 | Homo sapiens clone HQ0352 PRO0352 mRNA, |
| | | partial cds″ |
| | 630 | Homo sapiens genomic DNA; cDNA |
| | | DKFZp761G0924 (from clone DKFZp761G0924) |
| | 631 | Homo sapiens genomic DNA; cDNA |
| | | DKFZp434N2419 (from clone DKFZp434N2419) |
| | 632 | Homo sapiens hypothetical protein PRO2130 |
| | | (PRO2130), mRNA″ |
| | 633 | Homo sapiens cDNA FLJ11668 fis, clone |
| | | HEMBA1004705″ |
| | 634 | Homo sapiens cDNA FLJ11971 fis, clone |
| | | HEMBB1001208″ |
| | 635 | Homo sapiens cDNA FLJ12130 fis, clone |
| | | MAMMA1000251″ |
| | 636 | Homo sapiens cDNA: FLJ21527 fis, clone |
| | | COL05961″ |
| | 637 | Homo sapiens cDNA: FLJ21944 fis, clone |
| | | HEP04662″ |
| | 638 | Homo sapiens clone IMAGE: 297403, mRNA |
| | | sequence″ |
| | 639 | Synthetic construct Homo sapiens, clone |
| | | IMAGE: 3857181, mRNA″ |
| | 640 | Homo sapiens cDNA FLJ34300 fis, clone |
| | | FEBRA2006726″ |
| | 641 | Homo sapiens, clone IMAGE: 5440896, mRNA″ |
| | 642 | Homo sapiens cDNA clone IMAGE: 4793171 |
| | 643 | Homo sapiens cDNA clone IMAGE: 5285165 |
| | 644 | Homo sapiens cDNA clone IMAGE: 5301169 |
| | 645 | Homo sapiens mRNA; cDNA DKFZp686K13109 |
| | | (from clone DKFZp686K13109) |
| | 646 | Homo sapiens mRNA; cDNA DKFZp686J19109 |
| | | (from clone DKFZp686J19109) |
| | 647 | Homo sapiens cDNA FLJ26334 fis, clone HRT02648″ |
| | 648 | Homo sapiens cDNA FLJ45490 fis, clone |
| | | BRTHA2005831″ |
| NPM1 | 649 | Homo sapiens nucleophosmin 1, transcript variant 1, |
| | | mRNA″ |
| TP53 | 650 | Homo sapiens tumor protein p53, transcript variant 1, |
| | | mRNA″ |
| SEPT4 | 651 | Homo sapiens septin 4, transcript variant 1, mRNA″ |
| CPEB4 | 652 | Homo sapiens cytoplasmic polyadenylation element |
| | | binding protein 4, transcript variant 1, mRNA″ |

The candidate genes probes in Table 1 are hereinafter referred as “CM probes” or “the 652-gene transcription profiles.” In the following, all the statistical calculations are conducted through a processing module, which is a central processing unit (CPU). Specifically, the procedures of the present disclosure are described in detail below:

STEP 1. Construction of the Reference Gene Profiles for the Non-Cancer Tissue(s):

First, Step 1(a) is to extract the RNA expression levels of selected genes from the transcriptomic data derived from normal human tissues. Gene expression values from each organ were averaged from numerous persons in order to eliminate bias caused by single person. Therefore, 254 samples from thirty-nine different tissue origins are first selected from the datasets GSE1133, GSE2361 and GSE7307 to construct a training dataset. For this training dataset, the CEL files are acquired from GEO and then subjected to quality assessment by AffyQualityReport to remove poor quality arrays. The data passing quality-control is then subjected to the Robust Multichip Average (RMA, Irizarry R et al. Biostatistics 2003, 4(2):249-264) processing for data normalization. Both AffyQualityReport and RMA are obtained from the Bioconductor package in the R package. Following the standard preprocessing procedure, the transcriptomic data is subjected to further statistical and bioinformatics analyses.

Step 1(b) is to combine gene expression values for all the organs in test and build a gene-by-organ matrix as follows. The genes with high coefficient of variance across organs were selected for further analyses.

| | Gene | Organ | | | | | | |
|---|---|---|---|---|---|---|---|---|
| No. | Name | Liver | Lung | Breast | Colon | . . . | . . . | others |
| 1 | A | 2.3 | 2.3 | 1.3 | 0.5 | | | |
| 2 | B | 1.3 | 5.7 | 0.7 | 2.1 | | | |
| 3 | C | 4.1 | 0.4 | 1.3 | 5.0 | | | |
| . . . | . . . | . . . | . . . | . . . | . . . | . . . | . . . | . . . |

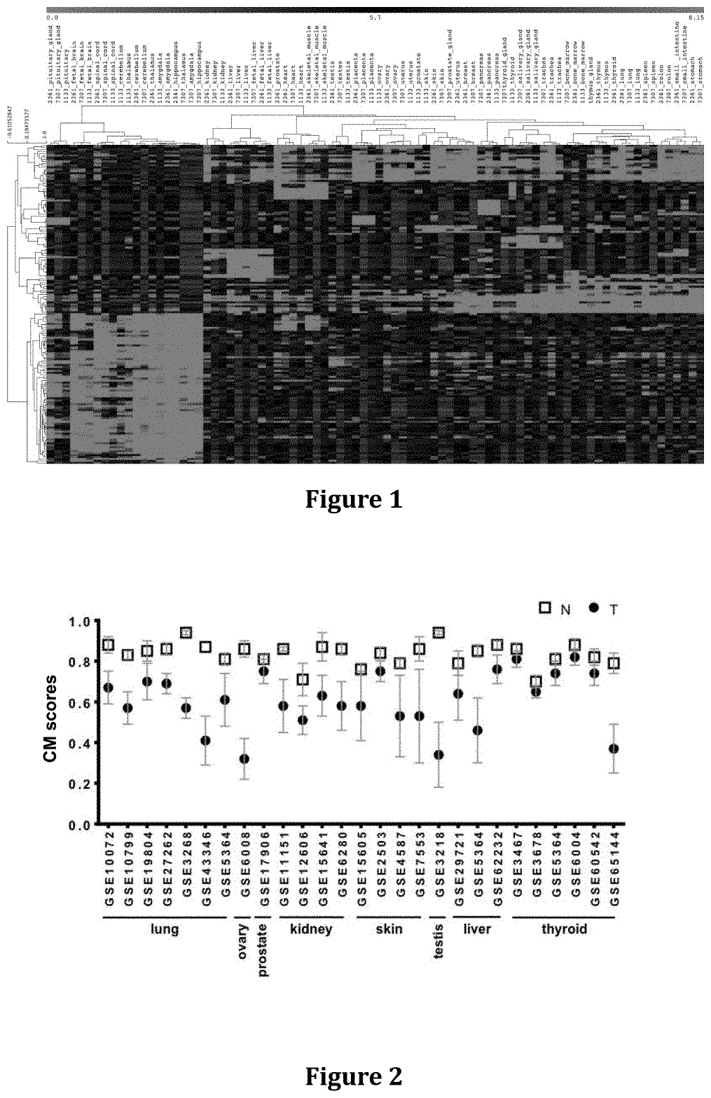

Step 1(c) is to perform a hierarchical clustering analysis with the gene-by-organ matrix to evaluate its effect on the tissue classification as FIG. 1 shows. Following the hierarchical cluster analysis, one representative gene for each cluster is selected and additional genes with highly similar expression profiles are removed. Such procedure results in the CM probes or the 652-gene transcription profiles as Table 1 shows.

The hierarchical cluster formula is as follows:

$$r = \frac{{\sum }_{i = 1}^{n} ( {X}_{i} - \overline{X} ) ( {Y}_{i} - \overline{Y} )}{\sqrt{{\sum }_{i = 1}^{n} {( {X}_{i} - \overline{X} )}^{2}} \sqrt{{\sum }_{i = 1}^{n} {( {Y}_{i} - \overline{Y} )}^{2}}}$$

Step 1(d) is to further validate tissue prediction by using independent datasets to make sure the expression profile of the selected genes adequately represents the designated organ at the normal state. Briefly, the expression values of the selected genes were extracted from each sample of the validation test to build an expression profile of the sample. The expression profile of the sample was then compared against the non-cancerous profiles from each of our collection of normal reference organs with an in-house program by computing the Pearson correlation coefficient between the sample profile and that from the non-cancer reference which was incorporated into the k-nearest neighbor (i.e., KNN) based tissue prediction program. The tissue with the highest coefficient of correlation (k=1) will be selected for the prediction.

The k-nearest neighbor formula is as follows:

$$S i m ( {d}_{i} , {d}_{j} ) = \frac{{\sum }_{k = 1}^{M} {W}_{i k} \times {W}_{j k}}{\sqrt{( {\sum }_{k = 1}^{M} {W}_{i k}^{2} ) ( {\sum }_{k = 1}^{M} {W}_{j k}^{2} )}}$$

Step 1(e) is to perform the repetitive gene-replacement in the reference list to improve the tissue classification until the outcome was satisfied. Any change in the constituent gene of the marker will result in a new run of reference profile construction. After completing all the above steps, the 652-gene transcription profile representing the organ at non-cancerous state is produced.

Again, it is worth noting that the tissue used in STEP 1(a) to 1(e) is a normal tissue with known organ but without any abnormal/disease tissue. Furthermore, in some embodiment, the said normal tissue with known organ can be extract or isolated from a subject (e.g., human) having or not having a cancer.

STEP 2. Measuring the Expression Levels of the “652-Gene Transcription Profile” in the Tumor Specimens in Test:

Step 2 (a) is to remove the tumor biopsy test sample from the patient and further extract the total RNA thereof through the currently available molecular biology technology.

Similar to STEP 1, Step 2 (b) is to determine the RNA expression level of the 652-gene transcription profile from the test sample in Step 2 (a) by applying the currently available molecular biology techniques (e.g., probe hybridization on a DNA microarray, hybridization on magnetic beads, rtPCR, or direct sequencing). Optionally, the expression level of the test sample can be further transformed into a list of numerical desire values representing the selected genes expression levels by applying a transforming process (e.g., data processing, data extraction and data re-formatting) and using a processing module (e.g., a central processing unit (CPU)).

STEP 3. Assessing the Pathological State of a Tumor Sample to Determine Whether it is a Normal/Benign or Malignant Tumor, or Whether it is a Primary or a Distantly Metastasized Tumor.

The similarity or dissimilarity (dissimilarity degree can be mathematically converted from a similarity degree) is measured on the expression levels of the selected genes between the sample tissue and the normal reference as described in STEP 1. In one embodiment, we use similarity score (e.g. the CM score). Further, because the CM score value is between 0 and 1, similarity or dissimilarity score can be calculated trough the following formula: (a) similarity degree=(CM score/1)*100; and (b) dissimilarity degree=1−similarity score. It is worth to know that the two subjects in comparison is identical when the similarity degree is 100%, and the two subjects in comparison is identical when the dissimilarity degree is 0%. However, the following two points are worth noting.

(1) These recorded expression values of genes were then subjected to computer processing which calculates the similarity between the sample gene profile and the reference gene profile to produce a CM score for the sample. The CM score here is based on the Pearson's correlation coefficient with the formula shown below:

$$r = \frac{n ( \sum xy ) - ( \sum x ) ( \sum y )}{\sqrt{[ n ( \sum {x}^{2} ) - {( \sum x )}^{2} ] [ n ( \sum {y}^{2} ) - {( \sum y )}^{2} ]}}$$

(Note: n indicated the number of genes used as the marker, x represents the gene expression values from the tested sample and y represents that from the reference.)

The calculation method (i.e., CM algorithm) for the similarity or distance between the expression profile from sample and that from reference is not limited to Pearson correlation. In some other embodiment, the method used to calculate the similarity or distance includes but are not limited to Spearman's rank correlation coefficient, Kendall, Mahalanobis distance, Euclidean distances, etc.

(2) Comparison of the CM score with the cutting score and the corresponding prediction is shown in Table 2 as follows.

TABLE 2

| CM score | Similarity | Dissimilarity | Prediction |
|---|---|---|---|
| >0.8 | >80% | <20% | Normal or benign tumor |
| 0.3-0.8 | 30-80% | 20-70% | Primary cancer |
| <0.3 | <30% | >70% | Distant metastatic cancer |

Further, the CM score is generated from the process of comparison in the Similarity-Based Mode and/or Distance-Based Mode. Specifically, in the Similarity-Based Mode, the higher the score is, the more similar the sample expression is to the “reference expression profile,” thereby inferring that the sample has a higher probability to be a benign or normal tissue. In the Distance-Based Mode, the higher the score is, the less similar the sample expression is to the “reference expression profile”, thereby inferring that the sample has a higher probability to be a malignant tumor.

Moreover, to classify whether the sample tissue is malignant or cancerous, the score is compared against the cut-off score which has been determined with either experimental or statistical methods (e.g. ROC, receiver's operation curve) or both.

For similarity-based scoring system, cut-offs A and B are established. Furthermore, score A is higher than score B. Score A provides significant sensitivities and specificities in separating primary cancer from normal tissue while score B provides significant sensitivities and specificities in separating primary cancer from metastatic cancer. In practice, if the sample score is lower than A but higher than B, the sample is predicted as a primary cancer; if the sample score is higher than A, the sample is predicted as a normal or benign tumor; and if the sample score is lower than B, the sample is predicted as a metastatic cancer.

For the distance-based scoring system, cut-offs C and D are established. Furthermore, score C is lower than score D. If the sample score is lower than D but higher than C, the sample is predicted as a primary cancer; if the sample score is lower than C, the sample is predicted as the normal or benign tumor; and if the sample score is higher than D, the sample is predicted as a metastatic cancer.

Accordingly, “the cells type identification method” in the present disclosure consists of three steps (i.e., STEP 1 to 3). First, STEP 1 is to generate the candidate genes (i.e., the CM probes or the 652-gene transcription profiles) listed in Table 1. Next, STEP 2 is to determine the expression of the candidate genes in the test sample. Finally, evaluate the CM scores of the test sample and then predict whether the cell type of the test sample is a normal cell/benign tumor cell, a primary tumor cell or a metastatic cell. As discussed above, the entire process/method of the present disclosure may be summarized to include the following steps: (1) Selecting candidate genes with high CV (coefficient of variance) from a normal sample without comparing to a disease sample, and the number of selected genes ranged from 20 to 652; (2) Validating the candidate genes expression with hierarchical clustering and tissue prediction; (3) Selecting the representative nucleotide fragments (e.g., for example, for the cDNA microarray, about 19 to 100 base pair long gene-specific fragments were designed for each selected gene and about 15 bases long oligonucleotides for primers of real time PCR) of the candidate genes according to the requirement of the RNA quantitation methods and further generating CM probes; (4) Determining the candidate genes expression level of a test sample by using the CM probes with the current available molecular biology techniques; (5) Calculating the CM score of the test sample based on the CM algorithm; (6) Predicting the cell type of the test sample based on the CM score.

In one embodiment, the present disclosure also provides a system used to develop a plurality of candidate probes to identify a cell type in a mammalian subject. Specifically, the system includes a detecting chip and a processing module, both of which are electrically connected to each other. The detecting chip contains a plurality of selected probes, which can bind a plurality of polynucleotide sequences selected from any one of SEQ ID No.1 to 652 or from any fragment of SEQ ID No.1 to 652, and detect a test sample array's expression level obtained from a mammalian subject that may or may not have a selected disease, disorder, genetic disorder. The processing module analyses the test sample array's expression level and further generates a score for the test sample. Further, the processing module can predict a cell type for the test sample based on the score of the test sample.

In one embodiment, the detecting chip used to identify the primary sites is a microarray chip or magnetic beads. In another embodiment, the processing module used to compare the plurality gene expressions or to develop the array containing the candidate probes is a central processing unit (CPU).

In one embodiment, the standard sample used to develop the selected probes includes blood, blood plasma, serum, urine, tissue, cells, organs, seminal fluids or any combination thereof. In another embodiment, the selected disease, disorder or genetic disorder includes hematologic malignancies or solid tumors.

### EXAMPLE 1

In the following, all the statistical calculations are conducted through a processing module, which is a central processing unit (CPU). The candidate genes probes (i.e., CM probes) used in Example 1 are narrow down to 50 or 56 genes selected from Table 1.

Materials and Methods

Tissues and Patients

Samples were collected with consent at the Tzuchi hospital in Hualian of Taiwan. Thirteen samples were obtained from thirteen patients who were subjected to surgical removal of the suspected malignant tumors in liver. Upon resection, tissue samples were immediately immersed into liquid nitrogen followed by RNAlater processing for later RNA extraction. The total RNA of normal liver from an Asian male adult was purchased from BioChain.

Microarray Hybridization

Total RNA extracted from the tumor samples with Quiagen RNAeasy was hybridized to the Affymetrix HG-U133 plus2.0 genechips following the manufacturer's standard protocol. Affymetrix HG-U133 plus2.0 contains 54,675 probe sets, representing around 38,572 unique UniGene clusters.

Datasets and Normalization

For the six GEO series to re-confirm the capability of the 56 genes “i.e, the CM probe” in characterizing a specific normal human organ/tissue, keyword search is carried out using the GEO database to generate a group of microarray datasets which were derived from Affymetrix GeneChip HG-U133 plus2.0 and composed of samples from both normal and cancerous tissues, that is, the first two of the five criteria described in the result session. The abstracts of those candidate GEO series were then read one by one in a random order to single out those qualified with the other three criteria described in the text. The search is stopped when the sixth qualified GEO series is found for the purpose of re-confirmation.

The test dataset used in Table 3 was constructed by pooling the six newly retrieved GEO series described above and the subset specific for cancer-study from the dataset previously used for large-scale validation analysis. The latter contained all the retrievable microarray data series (specified with prefix GSE in the GEO database) which were performed on the Affymetrix GeneChips HG133A or HG133plus2.0 and contained normal human samples from the twenty-four analyzable organs/tissue. The 24 normal tissues include kidney, skin, liver, lung, trachea, skeletal muscle, heart, bone marrow, thymus, pancreas, pituitary gland, salivary gland, placenta, uterus, ovary, prostate, skin, testis, amygdala, thalamus, cerebellum, spinal cord, fetal liver, fetal brain and thyroid.

All the GSE series used in this study with CEL files available were downloaded from the GEO website and were pre-processed with RMA in the Bioconductor package.

Assay Kit and Signal Detection

The QuantiGene assay kit was custom-made by Affymetrix Inc. upon the request by Mao-Ying Inc. Each sample was assayed in duplicates for confirmation and was processed following the standard protocol. At the end of each assay, the hybridization signals were detected with the Luminex® 100/200™.

Data Analysis/Tissue Prediction

The expression profiles of a designated gene set (the marker) had been constructed for each of the 24 normal organs/tissues as previously described. Briefly, the expression level of each gene of the marker was extracted from the whole-genome microarray data performed on normal human tissue of a designated organ. To see how similar a tissue specimen is to its normal counterpart, expression levels of the marker in the sample were also obtained from the sample for test. The Pearson's correlation coefficient (cf, equivalent to CM score in the present study) was then computed between these two lists of gene expression values. The Pearson correlation was carried out with a computer program implemented with the R language.

Statistical Analysis

The statistical analyses including standard deviation, P values of the student's t test were computed using the excel program. The P values of the student's t test in the Table 4 were calculated with parameters set at one tail and type 3.

Results

1. Consistent Transcription Profiles for the Normal Organs/Tissues

The tissue-prediction assays were repeated on several newly obtained datasets to re-confirm the previous disclosure by Hwang et al. Six datasets as shown in Table 3 were selected from the public database Gene Expression Omnibus (GEO, http://www.ncbi.nlm.nih.gov/geo/) with the following criteria:
(1) There were samples from both normal and cancerous tissues.
(2) The data were obtained from the experiments as performed on the Affymetrix GeneChips.
(3) There were specimens from the 24 types of organs/tissues, which were detectable by the CM algorithm.

TABLE 3
“Prediction of normal human organ/tissue by the 56-gene profiles”

| | | Number of | Number of | |
|---|---|---|---|---|
| | Number of | correct | wrong | |
| GSE | normal tissue | prediction | prediction | Tissue type |
| GSE15605 | 16 | 16 | 0 | Skin |
| GSE19804 | 59 | 59 | 0 | Lung |
| GSE27262 | 25 | 25 | 0 | Lung |
| GSE60542 | 30 | 30 | 0 | Thyroid |
| GSE62232 | 10 | 10 | 0 | Liver |
| GSE65144 | 13 | 13 | 0 | Thyroid |
| Total | 153 | 153 | 0 | |

The above six datasets of microarray experiments were used, including tissue samples from human skin, lung, thyroid and liver. Further, all 153 samples from normal organs/tissues in the six datasets were predicted correctly as Table 3 shows. This result is consistent with the previous finding, indicating that the expression profiles of the selected genes form the stable molecular features of a non-diseased human organ/tissue.

2. CM Profiles Differentiate Cancerous Tissues from Normal

A scoring system, the CM score, was designed which stands for “cancer malignancy score” reflecting the similarity/dissimilarity degree of the expression profile between the tested sample and the reference profile of the corresponding normal tissue. In the present disclosure, the CM score is equivalent to the correlation coefficient of Pearson's correlation. The Spearman's rank correlation coefficient was also tested and it showed the same result (data not shown).

In the past the tissue prediction tests usually provide less accuracy on cancerous tissues as compared to those on the normal tissues. Therefore, a test dataset was constructed based on the method and materials described above. The test dataset was made of transcriptomic data in twenty-seven independent GEO series derived from 927 cancerous and 340 normal samples covering kidney, liver, lung, ovary, prostate, skin, testis, and thyroid. Each array of the test dataset was computed for its CM score according to the procedure described previously. The higher the CM score is, the more the sample-in-test resembles its normal reference for the gene expression pattern.

To examine whether cancers are different from the normal on the 50 or 56 gene profiles, the average of the CM scores was taken for the group of cancer samples or the normal samples in each of the GSE datasets. As Table 4 discloses, it revealed that the averaged CM scores from the normal tissues were significantly higher than the cancers in all the tested GEO datasets, indicating a significant deviation of the cancer tissues from the normal for the overall expression profile of the marker genes. The averaged CM scores from the normal tissues were mostly above 0.80 with their standard deviations rarely going above 0.05, suggesting a good conservation of the expression pattern of the 56 genes in the normal tissue. Such expression pattern at a genomic level is tissue-specific and may be represented by a subset of the genes like the 56 genes for the 24 organs/tissues. This organ- or tissue-specific gene pattern is presented as a numerical formula among genes instead of the fold-change of overexpression or underexpression relative to a control gene.

In contrast, the averaged CM scores from the cancer distributed over a wider range and their deviations were higher than the normal. This phenomenon indicated that the overall gene expression pattern in the cancerous tissue was not similar to the normal reference. The wide range of the CM scores from a malignant tumor, indicating a big variety of gene expression patterns, may reflect the heterogeneous cancer cells in the tumor, an expected outcome of the multiple mutations existing in the cancer cells.

3. Difference Between Normal and Cancers Applied to Individual Samples

Though the cancer samples as a group exhibited significantly lower CM scores than their normal controls (see FIG. 2 and Table 4), it was not clear whether such difference was contributed by a small proportion of the tested samples or by the majority of them. We therefore sampled a few datasets from Table 4 to closely examine the CM scores of each individual sample. The datasets selected for such purpose included GSE10072 containing forty-nine normal and fifty-eight lung cancer samples, GSE15641 twenty-three normal and sixty-nine kidney cancer samples, GSE19804 sixty normal and sixty cancer samples, GSE6008 four normal and ninety nine ovary cancers, GSE62232 ten normal and eighty one liver cancer samples, and GSE65144 thirteen normal and twelve cancer samples.

TABLE 4

| | Numbers | | Numbers | | | |
|---|---|---|---|---|---|---|
| | of | | of | | | |
| | Cancer | CM scores | Normal | CM scores | t-Test | |
| Series_ID | Sample | (Tumor) | Sample | (Normal) | (p-value) | Note |
| GSE10072 | 58 | 0.67 ± 0.08 | 49 | 0.88 ± 0.04 | 3.08E−30 | Adenocarcinoma of lung |
| GSE10799 | 16 | 0.57 ± 0.08 | 3 | 0.83 ± 0.02 | 1.75E−09 | pulmonary |
| | | | | | | adenocarcinoma |
| GSE11151 | 62 | 0.58 ± 0.13 | 3 | 0.86 ± 0.01 | 4.28E−25 | various types of kidney |
| | | | | | | cancer |
| GSE12606 | 6 | 0.51 ± 0.07 | 4 | 0.71 ± 0.08 | 0.0143 | RCC |
| GSE15605 | 58 | 0.58 ± 0.17 | 16 | 0.76 ± 0.03 | 1.90E−11 | skin; cf (metastatic = 0.4; |
| | | | | | | primary = 0.62) |
| GSE15641 | 69 | 0.63 ± 0.1 | 23 | 0.87 ± 0.07 | 1.33E−19 | 5 types of kidney cancer |
| GSE17906 | 5 | 0.75 ± 0.06 | 5 | 0.81 ± 0.02 | 0.045 | Prostate cancer |
| GSE19804 | 60 | 0.7 ± 0.09 | 60 | 0.85 ± 0.05 | 2.69E−20 | Non-small cell lung cancer |
| GSE2503 | 5 | 0.75 ± 0.05 | 6 | 0.84 ± 0.03 | 0.011 | squamous cell carcinoma |
| GSE27262 | 25 | 0.69 ± 0.05 | 25 | 0.86 ± 0.03 | 1.36E−16 | stage I lung |
| | | | | | | adenocarcinoma |
| GSE29721 | 10 | 0.64 ± 0.13 | 10 | 0.79 ± 0.06 | 0.0025 | Hepatic cellular carcinoma |
| GSE3218 | 101 | 0.34 ± 0.16 | 5 | 0.94 ± 0.01 | 3.44E−62 | Various types of testis |
| | | | | | | cancer |
| GSE3268 | 5 | 0.57 ± 0.05 | 5 | 0.94 ± 0.01 | 1.09E−05 | Squamous cell lung cancer |
| GSE3467 | 9 | 0.81 ± 0.04 | 9 | 0.86 ± 0.03 | 0.0063 | Papillary Thyroid Cancer |
| GSE3678 | 7 | 0.65 ± 0.03 | 7 | 0.7 ± 0.03 | 0.00326 | Papillary Thyroid Cancer |
| GSE43346 | 23 | 0.41 ± 0.12 | 1 | 0.87 | N.A. | small cell lung cancer |
| GSE4587 | 9 | 0.53 ± 0.2 | 6 | 0.79 ± 0.02 | 0.00332 | Melanoma |
| GSE5364-liver | 9 | 0.46 ± 0.16 | 8 | 0.85 ± 0.03 | 1.04E−05 | Primary liver cancer |
| GSE5364-lung | 18 | 0.61 ± 0.13 | 12 | 0.81 ± 0.03 | 1.52E−06 | Primary lung cancer |
| GSE5364-thyroid | 35 | 0.74 ± 0.06 | 16 | 0.81 ± 0.03 | 1.82E−06 | Primary thyroid cancer |
| GSE6004 | 14 | 0.82 ± 0.04 | 4 | 0.88 ± 0.03 | 0.0055 | Papillary Thyroid Cancer |
| GSE6008 | 99 | 0.32 ± 0.1 | 4 | 0.86 ± 0.04 | 2.65E−07 | ovarian tumor: serous |
| GSE60542 | 35 | 0.74 ± 0.06 | 30 | 0.82 ± 0.04 | 4.11E−08 | papillary thyroid cancer |
| | | | | | | (PTC): primary tumors and |
| | | | | | | metastases |
| GSE62232 | 81 | 0.76 ± 0.07 | 10 | 0.88 ± 0.02 | 3.27E−16 | Hepatocellular carcinoma |
| GSE6280 | 14 | 0.58 ± 0.12 | 2 | 0.86 ± 0.03 | 0.0002 | various types of kidney |
| | | | | | | cancer |
| GSE65144 | 12 | 0.37 ± 0.12 | 13 | 0.79 ± 0.05 | 1.21E−08 | anaplastic thyroid |
| | | | | | | carcinoma |
| GSE7553 | 82 | 0.53 ± 0.23 | 4 | 0.86 ± 0.06 | 6.93E−06 | Various types of melanoma |
| Total | 927 | | 340 | | | |

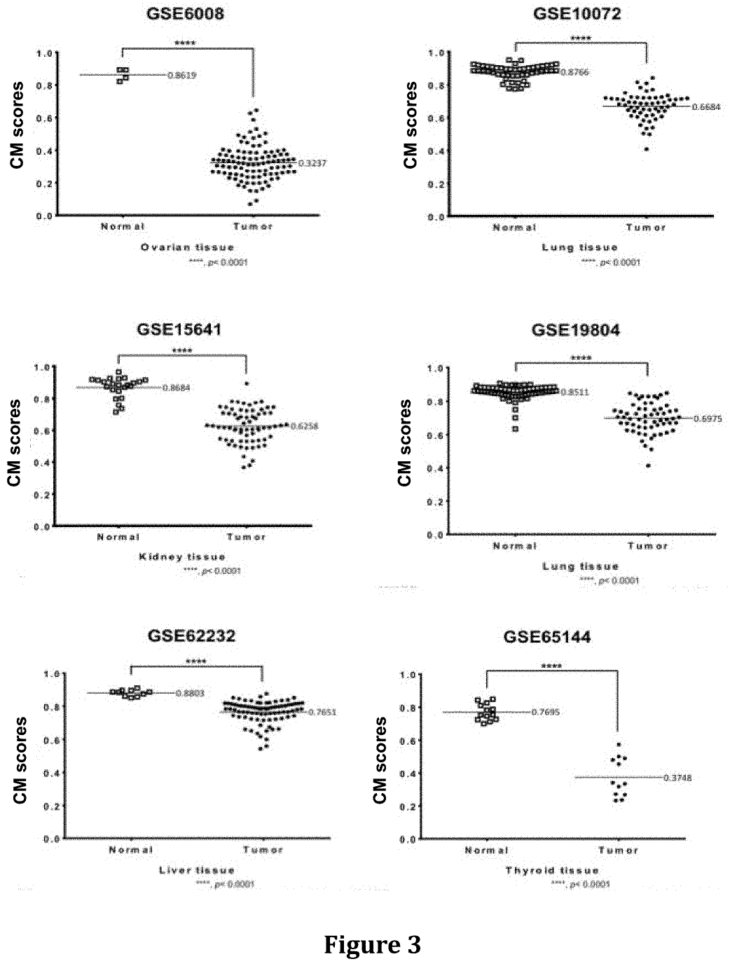

As FIG. 3 shows, the CM scores from each of the six analyzed datasets formed two major groups based on the CM score distributions, one higher group from the normal samples located in the higher CM score area and another lower group representing the cancer samples sitting at the lower CM score area. The two groups in all the tested datasets were so clearly separable that one could easily determine a cutting point of the score to differentiate the two types of tissues.

4. CM Score Worked Well with the Marker of Different Gene Combinations

To demonstrate that the CM score could differentiate cancers from non-cancers, meta-analysis was performed on four of the whole-genome gene-expression datasets acquired from GEO (e.g., Gene Expression Omnibus), which is a public database for gene expression. The criteria to select the datasets for test included firstly, the datasets should represent different organs, and secondly, the datasets should contain samples from both normal tissues and cancers. The datasets selected for such purpose are shown in Table 5 and include GSE10072 containing forty-nine normal samples and fifty-eight lung cancer samples, GSE11151 containing five normal samples and sixty-two kidney cancer samples, GSE6008 containing four normal samples and ninety nine ovary cancers, and GSE65144 containing thirteen normal samples and twelve thyroid cancer samples. Each data set was designated with the GEO accession number with a prefix GSE. The organs where the tumors were sampled were denoted in the parenthesis following the accession number of the dataset. Three combinations of genes were used as the markers to carry out the cancer/non-cancer discrimination. In addition to gene content, each of the three markers consisted of different number of genes, as indicated in Table 5.

Taking FIG. 3 for a reference, a cutting score at 0.8 was selected for each of four datasets to differentiate cancer from non-cancer tissue. A non-cancer (or normal) tissue would give CM score higher than 0.8 (i.e., similarity higher than 80% or dissimilarity lower than 20%) while a cancer tissue would provide a score lower than 0.8 (i.e., similarity lower than 80%, or dissimilarity higher than 20%). The sensitivities (Sensitivity=true positives/(true positive+false negative)) and specificities (Specificity=true negatives/(true negative+false positives)) of the four datasets were computed and the results are shown in Table 5: the accuracies, sensitivities and specificities for all the four datasets were all high.

According to the results of FIG. 3 and Table 5, it can be concluded: (1) the CM score difference which had been observed at the large-scale analysis (see Table 4) was contributed by the majority of individual samples in analysis instead of by a proportion of “significant”-valued samples; (2) the malignant tumors did exhibit significant difference in their global gene expression pattern from their mother organ; and (3) such feature could have a great potential to be developed into an objective cancer diagnostics in the majority of individual cases to facilitate diagnosis of cancers.

It appears in Table 5 that a score around 0.8 (i.e., similarity around 80% or dissimilarity around 20%) worked well to separate cancer and normal tissues in various organs, except thyroid.

Regarding the small overlaps between the CM score distributions of normal and that of cancer, it can be attributed to false positives and false negatives. For example, perhaps the normal samples (i.e., false positives) at the overlapping area were contaminated with the adjacent cancer cells, or the tumor content in the cancer sample was too low to be observed under microscope but sufficient to be picked up by molecular hybridization. One possibility for false negatives is that it may be out of the detection scope of the CM score to differentiate certain subtypes of cancers from their originated normal tissue.

5. Applications of CM Probes to Clinical Samples

In order to learn how the CM scores may relate to the status of the cancers, the CM analysis was applied directly to clinical specimen through collaborating with surgical oncology department of Tzuchi Hospital in Hualian, Taiwan. Tissue samples of malignant tumors were obtained with informed consent from patients who had been diagnosed with cancer and subjected to resection at Tzuchi hospital. To expand the group of normal tissue, an RNA sample from “normal” liver purchased from BioChain Inc. was also included, producing a total of 27 samples consisting of 16 liver tumors, 7 normal livers, 2 pancreatic tumors, 1 thyroid tumor, and 1 normal thyroid specimen. Total RNA was extracted from each specimen following a standard protocol, and, after discarding unsuitable samples using a process of RNA quality control, the RNA was hybridized to arrays of Affymetrix HU133 plus2.0 GeneChip.

TABLE 5
“The sensitivities and specificities of normal/cancer separation when CM score
was set at 0.8 using different gene combinations as the cancer markers”

| | GSE10072 | | GSE6008 | | GSE11151 | | GSE65144 | |
|---|---|---|---|---|---|---|---|---|
| Marker | (Lung) | | (Ovary) | | (Kidney) | | (Thyroid) | |
| (cutoff) | Sensitivity | Specificity | Sensitivity | Specificity | Sensitivity | Specificity | Sensitivity | Specificity |
| 26 (0.8) | 100% | 92% | 100% | 100% | 100% | 100% | 100% | 69% |
| 29 (0.8) | 93% | 90% | 100% | 100% | 100% | 100% | 71% | 100% |
| 36 (0.8) | 86% | 98% | 100% | 100% | 100% | 100% | 100% | 69% |

The CM score was first computed for each sample. The corresponding pathological data from each patient was retrieved from the files at the hospital and was organized with the CM scores to produce the results in Table 6. The majority of normal samples exhibited a CM score of 0.79 or higher, whereas almost all the tumors exhibited CM scores lower than 0.81. The only tumor sample with a CM score significantly higher than 0.81 was sample (#100T), whose donor exhibited only very mild symptoms of liver cancer. Additionally, the liver cancer of patient (#100T) was classified as BCLC-A, indicating an early stage hepatocellular carcinoma. On the other hand, the normal sample #87 exhibited a CM score of 0.68, the lowest among all the normal specimens tested. Its matched tumor sample (#88T) happened to be included in this study and also exhibited the lowest CM score (0.55) among the 13 primary hepatocellular carcinoma (HCC) samples. The pathological report of sample (#88T) described a relatively severe malignancy compared with other HCC specimens. In summary, these results suggested a positive correlation between CM score value and tumor malignancy. It should be noted that the “normal” samples here, unlike normal references from non-diseased donors, were peripheral tissues of the organ with cancer. Therefore, it was not surprising that the CM scores of the normal samples did not exhibit all CM scores as high as those of healthy individuals.

Among the 27 samples, four of the tumor samples gave especially low CM scores, including three diagnosed as cholangiocarcinoma (sample #8T, #16T, and #386T) and one (sample #206T) as a solid pseudopapillary neoplasm of pancreatic cancer. These can be explained after considering that reference the 652-gene transcription profiles represent the gene expression status of normal tissue and that low CM scores indicate dissimilarity to this reference. Thus, although cholangiocarcinomas are found in the liver, they originate from the bile duct and so, by nature, are highly dissimilar to liver tissue and so exhibit very low CM scores when compared with the 652-gene transcription profile of normal liver. The solid pseudopapillary neoplasm of pancreatic cancer was an unusual form of pancreatic carcinoma and was the result of cell death induced by necrosis. The morphology and function of such a tumor, therefore probably only distantly resemble that of normal pancreas tissue, thereby leading to a low CM score when compared with normal pancreas.

Thus the results supported the hypothesis of the present disclosure.

6. CM Score may Relate to Degrees of Malignancies of a Tumor

CM scores are also observed to possibly correlate with the degree of the malignancies of the tumor. For example, there are four datasets of skin cancer listed on Table 4. Three of them (i.e., GSE15605, GSE4587, and GSE7553) contained samples from melanoma, a highly aggressive and deadly type of skin cancer, while the other one GSE2503 from the squamous skin cancer which is mild compared with melanoma. The CM scores for the skin cancers in GSE2503 were higher than those from the melanoma in the other three datasets. Among the seven datasets from lung cancer, the lowest CM score occurred with small cell lung cancer, a quickly spreading and highly aggressive subtype of lung cancer compared to other subtypes. Similarly, among the six GEO series from the thyroid cancer, five of them from papillary thyroid cancer had CM scores nearly as high as those from their normal controls. The papillary thyroid cancer is the most common type of thyroid cancer and is known to be well-differentiated, slow-growing, and with good prognosis. While the GSE 65144 from anaplastic thyroid carcinoma is with a low CM score (0.37±0.12) for the cancer samples. The anaplastic thyroid carcinoma is a very aggressive but rarely found subtype of thyroid cancer. It has very poor prognosis and is resistant to most treatments. Taken together, the CM scores derived from these clinical specimens correlate with the cancer progression.

7. Validation of CM Scores-Gene Marker on Magnetic Beads with Clinical Samples

TABLE 6
“The cancer characterization of clinical samples from Tzuchi hospital for microarray analysis”

| Sample ID | Organ | ref_organ | CM score | Diagnosis | Pathological_report |
|---|---|---|---|---|---|
| commercial | liver | liver | 0.85 | | commercial product, from a |
| | | | | | 60-year-old Asian male |
| | | | | | (BioChain, cat. no: |
| | | | | | R1234149-50-D01) |
| 263N | liver | liver | 0.86 | | well encapsulated, |
| | | | | | angiolymphatic invasion |
| 337N | liver | liver | 0.79 | distant normal of | mild tumor necrosis, partially |
| | | | | primary liver cancer | encapsulated with focal |
| | | | | | infiltrative border |
| 353N | thyroid | thyroid | 0.83 | | non-toxic multinodular goiter |
| 373N | liver | liver | 0.84 | | tumor necrosis, partially |
| | | | | | encapsulated with focal |
| | | | | | infiltrative border, |
| | | | | | angiolymphatic invasion |
| 393N | liver/cholangio- | liver | 0.82 | distant normal liver | mass-forming tumor growth; |
| | carcinoma | | | tissue of | non-tumoral liver tissue: non- |
| | | | | cholangiocarcinoma | cirrhotic |
| 87N | liver | liver | 0.68 | | mild tumor necrosis, |
| | | | | | angiolymphatic invasion, |
| | | | | | portal/hepatic vein |
| | | | | | thrombosis, non-encapsulated |
| | | | | | with infiltrative border |
| 99N | liver | liver | 0.87 | | non-encapsulated with |
| | | | | | infiltrative border |
| 206T | pancreas | pancreas | 0.21 | solid | tumor is confined to pancreas |
| | | | | pseudopapillary | |
| | | | | neoplasm of | |
| | | | | pancreatic cancer | |
| 16T | cholangiocarcinoma | liver | 0.32 | cholangiocarcinoma | cholangiocarcinoma; |
| | | | | | angiolymphatic invasion |
| 8T | cholangiocarcinoma | liver | 0.3 | cholangiocarcinoma | mild tumor necrosis, |
| | | | | | angiolymphatic invasion, |
| | | | | | non-encapsulated with |
| | | | | | infiltrative border |
| 386T | cholangiocarcinoma | liver | 0.4 | cholangiocarcinoma | mass-forming tumor growth, |
| | | | | | lymphatic vascular invasion |
| | | | | | (small vessel) |
| 88T | liver | liver | 0.55 | | mild tumor necrosis, |
| | | | | | angiolymphatic invasion, |
| | | | | | portal/hepatic vein |
| | | | | | thrombosis, non-encapsulated |
| | | | | | with infiltrative border |
| 340T | liver | liver | 0.67 | primary liver cancer | poorly differentiated, marked |
| | | | | | tumor necrosis, non- |
| | | | | | encapsulated with infiltrative |
| | | | | | border, angiolymphatic |
| | | | | | invasion, portal/hepatic vein |
| | | | | | thrombosis |
| 330T | liver | liver | 0.77 | primary liver cancer | mild tumor necrosis, partially |
| | | | | | encapsulated with focal |
| | | | | | infiltrative border, |
| | | | | | angiolymphatic invasion, |
| | | | | | portal/hepatic vein |
| | | | | | thrombosis |
| 400T | liver | liver | 0.74 | primary liver cancer | well differentiated; mild |
| | | | | | tumor necrosis, |
| | | | | | angiolymphatic invasion |
| | | | | | (positive in capsule), partially |
| | | | | | encapsulated with focal |
| | | | | | infiltrative border |
| 40T | liver | liver | 0.72 | primary liver cancer | tumor necrosis, |
| | | | | | angiolymphatic invasion, |
| | | | | | partially encapsulated with |
| | | | | | focal infiltrative border |
| 60T | pancrease head | pancrease | 0.72 | primary pancreatic | chronic pancreatitis with |
| | | | | cancer | massive fibrosis |
| 104T | thyroid | thyroid | 0.78 | benign thyroid | adenomatous goiter |
| | | | | tumor | |
| 36T | liver | liver | 0.76 | primary liver cancer | angiolymphatic invasion, |
| | | | | | partially encapsulated with |
| | | | | | focal infiltrative border |
| 30T | liver | liver | 0.78 | primary liver cancer | Well encapsulated with focal |
| | | | | | capsular invasion, angio- |
| | | | | | lymphatic invasion |
| 122T | liver | liver | 0.76 | primary liver cancer | partially encapsulated with |
| | | | | | focal infiltrative border, |
| | | | | | angiolymphatic invasion |
| 50T | liver | liver | 0.81 | primary liver cancer | moderate tumor necrosis, |
| | | | | | angiolymphatic invasion, |
| | | | | | portal/hepatic vein |
| | | | | | thrombosis, non-encapsulated |
| | | | | | with infiltrative border |
| 44T | liver | liver | 0.78 | primary liver cancer | angiolymphatic invasion, |
| | | | | | well encapsulated with focal |
| | | | | | capsular invasion |
| 384T | liver | liver | 0.82 | primary liver cancer | angiolymphatic invasion |
| 6T | liver | liver | 0.8 | primary liver cancer | angiolymphatic invasion, |
| | | | | | partially encapsulated with |
| | | | | | focal infiltrative border |
| 100T | liver | liver | 0.85 | primary liver cancer | non-encapsulated with |
| | | | | | infiltrative border |

According to Table 5 and Table 6, the cutoff CM score is implied to be around 0.8 to separate cancer from non-cancer and above 0.2 to discern primary from metastatic if using the Affymetrix microarrays for the mRNA quantitation. It is curious whether the same cutoff values may also be applicable if applying a different technological platform, such as magnetic beads. For verification, clinical specimens on the magnetic bead system were tested with the Quantigene plex 2.0, carried by the Affymetrix Inc. Tumor specimens were obtained from 32 patients who suffered from cancers at different organs including breast, colon, liver and pancreas (as Table 7 shows). The total RNA from the samples was hybridized to the probes of the 50 or 56 gene marker which had been pre-conjugated onto the magnetic beads. The output expression levels of each of the marker genes from individual specimens were computed to come up with the CM scores following the routine computational procedure described herein. It was found that all the primary cancer gave a score below 0.8 (i.e., below similarity 80%, or above dissimilarity 20%). When applying 0.2 (i.e., similarity 20% or dissimilarity 80%) as the cutoff value to differentiate primary from metastatic cancers, 100%, 95%, and 97% were obtained for sensitivity, specificity and accuracy, respectively (as Table 8 shows). The results agreed with the analyses of Table 6. The result showed that the score about 0.2 to 0.3 (i.e., similarity 20-30% or dissimilarity 70-80%) could work well as the cutoff on separation of primary cancer from metastatic cancers while RNA quantitation was performed on magnetic beads.

TABLE 7
“Summary of the clinical samples used
in the magnetic bead experiments”

| Anatomic site | Primary | Metastatic | Total |
|---|---|---|---|
| liver | 12 | 9 | 21 |
| colon | 6 | 0 | 6 |
| breast | 4 | 0 | 4 |
| pancreas | 0 | 1 | 1 |

TABLE 8
“CM score threshold at 0.2 can well discern metastatic
cancer from primary cancer when performing mRNA quantitation
on the magnetic beads”

| | Prediction | | |
|---|---|---|---|
| Diagnosis | >0.2 (Primary) | <0.2 (Metastatic) | Sensitivity: 100% |
| Primary | 21 | 1 | Specificity: 95% |
| Metastatic | 1 | 10 | Accuracy: 97% |

8. Benign Tumors Gave High CM Scores

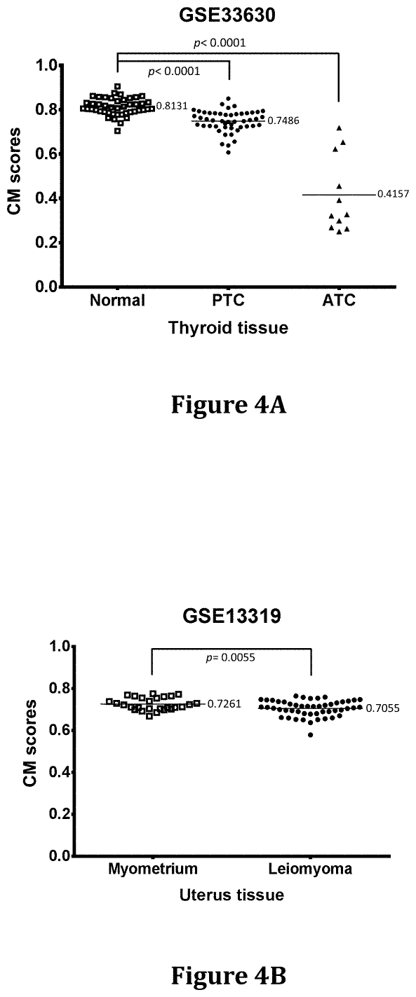

The papillary thyroid cancer (i.e., PTC), the common subtype of thyroid cancer, often exhibits quite benign characteristics: well-differentiated, slow growing, unlikely to invade blood vessels, good prognosis after treatment scores etc. As FIG. 4A shows, the CM scores of the PTC samples appeared quite close to those of the normal, reflecting the benign characteristics. While the anaplastic thyroid cancer (i.e., ATC), the aggressive subtype of thyroid cancer, showed significantly lower scores than either normal or PTC. It should be noted that the encapsulated follicular variant of papillary thyroid carcinoma (EFVPTC) has recently been reclassified and renamed into “non-invasive follicular thyroid neoplasm with papillary-like nuclear features” (NIFTP) to better reflect its biological and clinical characteristics to avoid over-treatment of the patients following an international, multidisciplinary and retrospective study. (Yuri E. Nikiforov, M D, PhD; Raja R. Seethala, MD; Giovanni Tallini, MD et al. JAMA Oncol. 2016; 2(8):1023-1029. doi:10.1001/jamaoncol.2016.0386).

Similar results were observed in other cancers. When applying the method of the present disclosure to the datasets (e.g., GSE13319) which contained benign tumors leiomyoma and the normal tissue myometrium of uterus, the CM scores from these two categories basically overlapped with each other as FIG. 4B shows, indicating the non-cancerous nature of the benign tumors. GSE13319 contained data from 50 samples of leiomyoma, benign tumors of uterine, in addition to 27 samples of the myometrium, the middle layer of a uterine. Following the expression profile analysis, the CM score distribution from leiomyoma almost overlapped with those for the myometrium. The averaged CM score for leiomyoma (0.71±0.04) and myometrium (0.73±0.03) were rather close to each other.

In summary, the present disclosure shows that a gene-based novel procedure was established for cancer diagnosis with five combinations of gene sets on two different experimental systems, using a high density gene expression microarray and a magnetic-bead assisted multi-gene expression system. This procedure returned a score, e.g., a CM score, by comparing the expression profile of selected genes (marker) from the specimen-in-test to that of a normal reference. The score in this example was the Pearson's correlation coefficient. There are two thresholds: the higher threshold at around 0.8 (i.e., the higher similarity threshold at around 80% or the lower dissimilarity threshold at 20%) and the lower at around 0.2 to 0.3 (i.e., the lower similarity threshold at around 20-30%, or the higher dissimilarity at around 70-80%). The tissue with CM score higher than the higher threshold would very likely be a normal tissue or benign tumor; lower than the first threshold but higher than the second would likely be a primary cancer; lower than the second threshold would likely be a metastatic cancer.

I/We claim:

1. A method for developing a plurality of candidate probes to identify a cell type in a mammalian subject, comprising:
(a) generating, with a detecting chip, a plurality of gene expressions for a standard sample of a mammalian subject,
wherein the standard sample is a cell of a known tissue;
(b) comparing, with a processing module, the plurality of gene expressions to generate a comparison result; and
(c) developing, based on the comparison result, an array containing a plurality of selected probes, wherein the plurality of selected probes can bind a plurality of polynucleotide sequences selected from any one of SEQ ID No.1 to 652 or from any fragment of SEQ ID No.1 to 652,
wherein the detecting chip is electrically connected to the processing module.

2. The method according to claim 1, wherein a number of the plurality of selected probes is about 200.

3. The method according to claim 1, wherein a number of the plurality of selected probes is about 100.

4. The method according to claim 1, wherein a number of the plurality of selected probes is about 50-60.

5. The method according to claim 1, wherein a number of the plurality of selected probes is about 25-35.

6. The method according to claim 1, wherein a length of the plurality of selected probes is at least 15 nucleotides.

7. The method according to claim 1, wherein the standard sample is not diagnosed with a selected disease, disorder, genetic disorder or any combination thereof.

8. The method according to claim 1, wherein the mammalian subj ect is diagnosed with a selected disease, disorder, genetic disorder or any combination thereof.

9. The method according to claim 1, wherein the standard sample is blood, blood plasma, serum, urine, tissue, cells, organs, seminal fluids or any combination thereof.

10. The method according to claim 1, wherein step (b) does not include: comparing the plurality of gene expressions for the standard sample with an abnormal sample of a subject diagnosed with a selected disease, disorder, genetic disorder or any combination thereof.

11. The method according to claim 1, wherein in step (c), the array is developed by applying the following: Pearson's correlation, Spearman's rank correlation, Kendall, k-means, Mahalanobis distance, Hamming distance, Levenshtein distance, Euclidean distances or any combination thereof.

12. The method according to claim 1, wherein step (c) further includes:
(c1) analyzing a correlation factor between an expression of a selected sequence of the plurality of the selected probes and an expression of the plurality of polynucleotide sequences selected from any one of SEQ ID No.1 to 652 or from any fragment of SEQ ID No.1 to 652.

13. The method according to claim 12, wherein the correlation factor includes binding affinity.

14. A method for characterizing a cell type in a mammalian subject, comprising:
(a′) detecting, with a detection chip that contains the plurality of selected probes as in any one of claims 1-5, an expression level of a test sample array obtained from a mammalian subject diagnosed with a selected disease, disorder, genetic disorder,
wherein a plurality of selected probes can bind the plurality of polynucleotide sequence selected from any one of SEQ ID No.1 to 652 or from any fragment of SEQ ID No.1 to 652 as in any one of claims 1-5;
(b′) analyzing, with a processing module, the test sample based on the detected expression level to generate a score for the test sample; and
(c′) predicting, with the processing module, a cell type for the test sample based on the score for the test sample.

15. The method according to claim 14, wherein the score for the test sample is calculated based on a similarity or dissimilarity degree.

16. The method according to claim 15, wherein the cell type for the test sample is characterized as a normal/benign tumor cell when the similarity degree is >about 80%.

17. The method according to claim 15, wherein the cell type for the test sample is characterized as a primary tumor cell when the similarity degree is about 30-80%.

18. The method according to claim 15, wherein the cell type for the test sample is characterized as a metastatic tumor cell when the similarity degree is <about 30%.

19. The method according to claim 15, wherein the cell type for the test sample is characterized as a normal/benign tumor cell when the dissimilarity degree is <about 20%.

20. The method according to claim 15, wherein the cell type for the test sample is characterized as a primary tumor cell when the dissimilarity degree is about 20-70%.

21. The method according to claim 15, wherein the cell type for the test sample is characterized as a metastatic tumor cell when the dissimilarity degree is >about 70%.

22. The method according to claim 14, wherein the selected disease, disorder or genetic disorder includes hematologic malignancies or solid tumors.

23. The method according to claim 14, therein in step (b′), the score is generated by applying the following: Pearson's correlation coefficient, Spearman's rank correlation coefficient, Kendall, Mahalanobis distance, Euclidean distances or any combination thereof.

24. The method according to claim 14, wherein the detecting chip includes a microarray, a next-generation sequencing device, a quantitative polymerase chain reaction (i.e., qPCR) and magnetic beads.
